(19) United States
(12) Patent Application Publication
(10) Pub. No.: US 2003/0170205 A1
(43) Pub. Date: Sep. 11, 2003

(54) Interleukin-1 homolog zil1a7

(76) Inventor: Paul O. Sheppard, Granite Falls, WA (US)

Correspondence Address: Gary E. Parker, ZymoGenetics, Inc., 1201 Eastlake Avenue East, Seattle, WA, 98102, US

(21) Appl. No.: 10/132,113

(22) Filed: Apr. 24, 2002

Related U.S. Application Data
(60) Provisional application No. 60/286,481, filed on Apr. 25, 2001.

(51) Int. Cl. A61K 38/20; C07H 21/04; C12P 21/02; C12N 5/06; C07K 14/54

(52) U.S. Cl. 424/85.2; 435/69.52; 435/320.1; 435/325; 530/351; 536/23.5

(57) ABSTRACT

Homologs of interleukin-1, materials and methods for making them, compositions comprising them, and methods of using them are disclosed. The homologs are proteins comprising a sequence of amino acid residues as shown in SEQ ID NO:2 from residue 32 through residue 166. The proteins have inflammation modulating activity and are useful within related research and therapeutic applications.

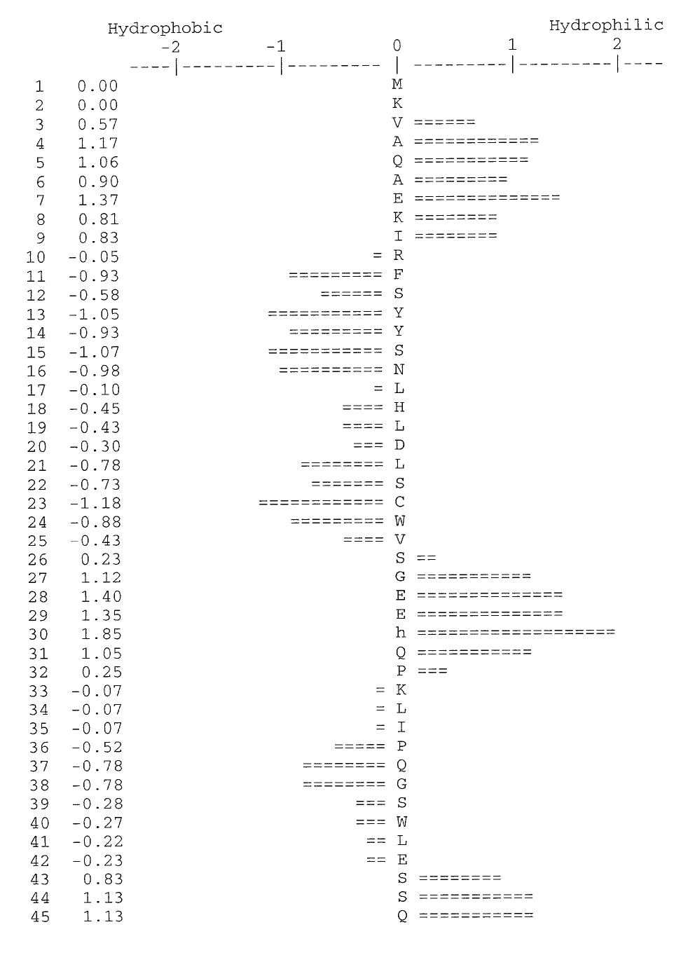

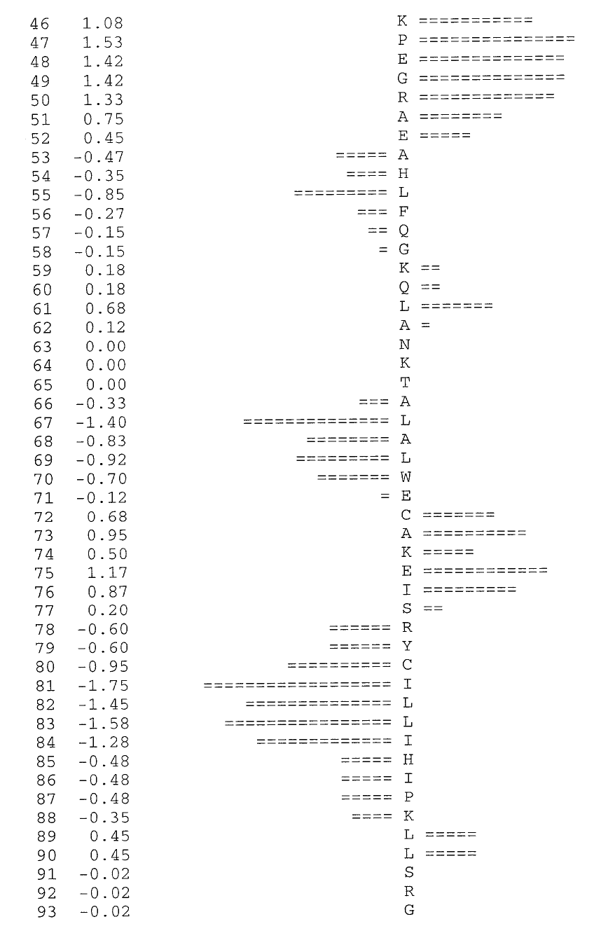

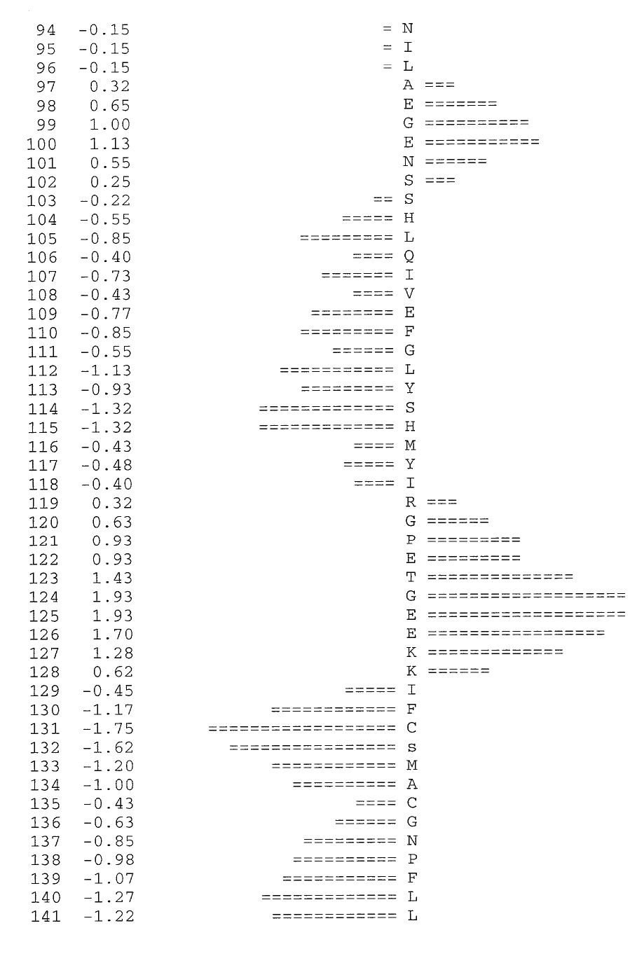

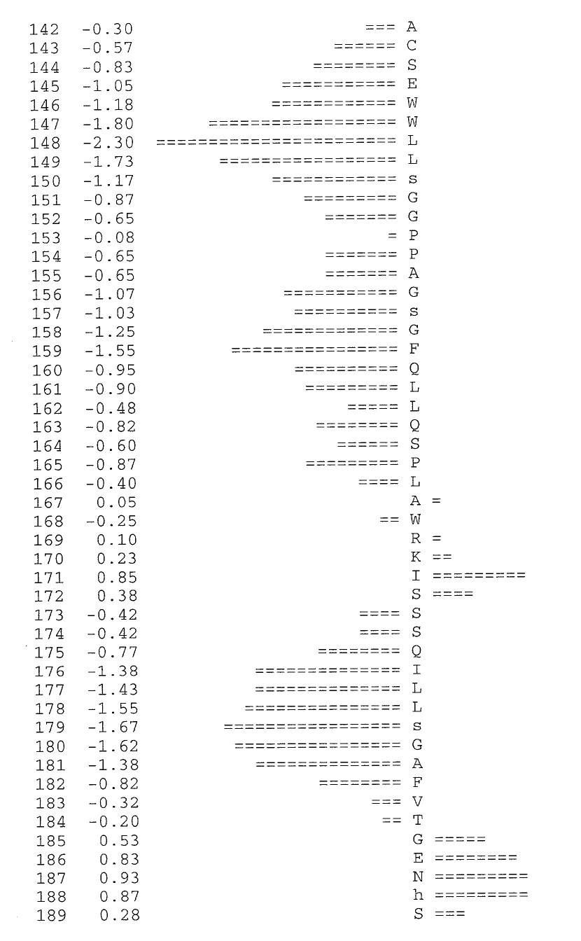

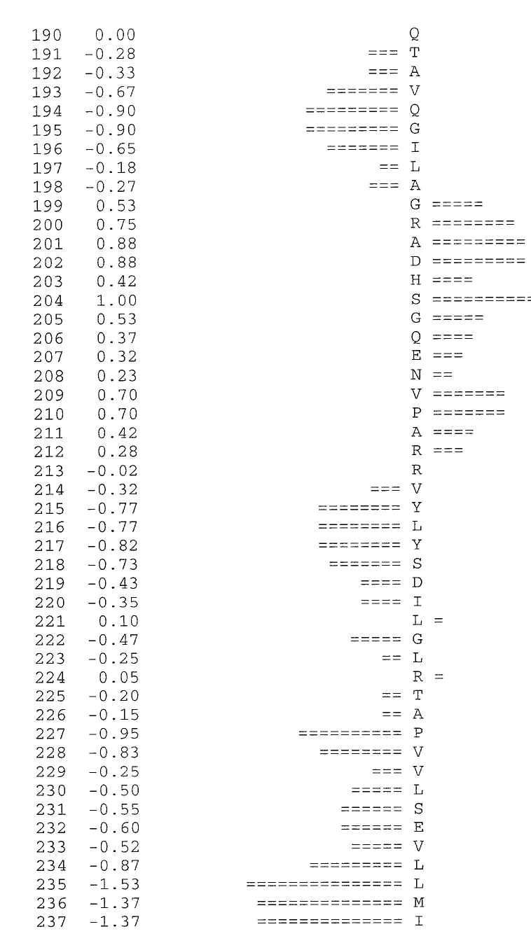

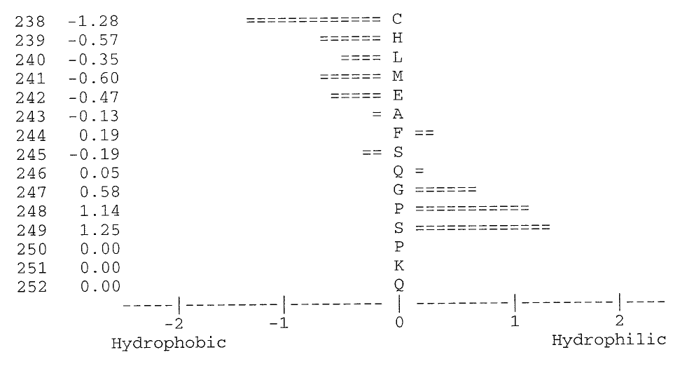

### CROSS-REFERENCE TO RELATED APPLICATION

This application claims the benefit under 35 U.S.C. § 119(e) of provisional application No. 60/286,481, filed Apr. 25, 2000.

### BACKGROUND OF THE INVENTION

In multicellular organisms, cell growth, differentiation, migration, and metabolism are controlled by a variety of polypeptide factors. These factors regulate both normal development and pathogenesis.

Two structurally related families of cytokines are the interleukin-1 (IL-1) family and the fibroblast growth factors (FGFs). Although the amino acid sequence identity is rather low among family members and between the two families, members of both families share a common higher-order structure. This structure can be envisioned as three 4-stranded covalent monomers assembled into a 12-stranded structure (a “trefoil”).

Members of the IL-1 family of cytokines mediate immune and inflammatory responses. The three known members of this family share recognizable sequence homology and common receptor binding activities. IL-1α and IL-1β are pro-inflammatory cytokines, while the third family member, IL-1 receptor antagonist (IL-1ra), is an antagonist of IL-1α and IL-1β activities. IL-1ra is unusual in that it is the only known, naturally occuring cytokine receptor antagonist with no apparent agonist function. The ability of IL-1ra to bind, but not activate, the IL-1 receptor suggests that IL-1ra is a negative regulator of inflammation (Dripps et al., J. Biol. Chem. 266:10331-10336, 1991; Granowitz et al., Blood 79:2356-2363, 1992).

The interleukins mediate a variety of inflammatory pathologies. At low concentrations IL-1α and IL-1β act locally on mononuclear phagocytes and the vascular endothelium to induce further IL-1 and IL-6 synthesis. IL-1 does not act directly on leukocytes and neutrophils, but causes the mononuclear phagocytes and endothelial cells to activate leukocytes. When secreted in large quantities into the bloodstream, IL-1 has endocrine effects, including fever, synthesis of acute phase plasma proteins, and cachexia.

Early reports described naturally occurring inhibitors of IL-1 activity in many sources: urine from febrile patients (Seckinger et al., J. Immunol. 139:1546-1549, 1987; Mazzei et al., Eur. J. Immunol. 20:683-689, 1990), monocytic leukemia patients (Seckinger et al., ibid.), or juvenile chronic arthritis patients (Prieur et al., Lancet 2:1240-1242, 1987); synovial fluid of rheumatoid arthritis patients (Lotz et al., J. Clin. Invest. 78:713-721, 1986); and viral-infected monocytes or B-cells (Scala et al., J. Exp. Med. 159:1637-1652, 1984). These inhibitory bioactivities appear to be heterogeneous with a range of molecular mass from 18 to 67 kDa, but they remain largely uncharacterized. The first demonstration of a true IL-1 receptor antagonist came from the purification and cloning of recombinant IL-1ra from human peripheral blood mononuclear cells (PMNC) cultured on IgG-coated plates and from the human monocytic cell line U937 activated with phorbol ester (PMA) (Carter et al., Nature 344:633-638, 1990; Hannum et al., Nature 343:336-340, 1990; Eisenberg et al., Nature 343:341-346, 1990).

Serum from healthy people has a low level of circulating IL-1ra activity (200-400 pg/ml). Serum IL-1ra levels are dramatically increased in patients with acute or chronic inflammatory disease, certain cancers, infectious diseases, and septic shock (Fisher et al., Blood 79:2196-2200, 1992), major surgery for Hirshsprung's disease (O Nuallain et al., Clin. Exp. Immunol. 93:218-222, 1993), liver disease (Sekiyama et al., Clin. Exp. Immunol. 98:71-77, 1994), Hodgkin's disease (Gruss et al., Lancet 340:968, 1992), and colitis (Hyams et al., Dig. Dis. Sci. 39:1893-1899, 1994). Other acute and chronic disorders also induce local production of IL-1ra, for example, rheumatoid arthritis (Firestein et al., J. Immunol. 149:1054-1062 1992), Lyme arthritis (Miller et al., Lancet 341:146-148, 1993), and nonperforating Crohn's disease (Gilberts et al., Proc. Natl. Acad. Sci. USA 91: 12721-12724, 1994). Induction of endotoxemia in human volunteers induces 100-fold excesses of IL-1ra over IL-1β in serum, however this level is still apparently insufficient to abolish IL-1 activity in vivo (Granowitz et al., Lancet 338:1423-1424, 1991). Numerous experiments have shown the potential for systemic, intravenous injection of IL-1ra to attenuate the effects of administered IL-1 or LPS in animal models of endotoxemia or synovitis (Dinarello and Thompson, Immunol. Today 12:404-410, 1991). When antibodies to IL-1ra were administered iv in a rabbit formalin-induced colitis model there was significant exacerbation of intestinal inflammation and mortality (Ferretti et al., J. Clin. Invest. 94:449-453, 1994).

IL-1ra has been investigated for use in treating several chronic inflammatory disorders including rheumatoid arthritis (Henderson et al., Cytokine 3:246-249, 1991), chronic myelogenous leukemia (CML) (Schiro et al., Blood 83:460-465, 1994), and inflammatory bowel disease (IBD) (Cominelli et al., Gastroenterology 103:65-71, 1992). Some evidence also suggests that IL-1ra may be useful in the treatment of psoriasis. Normal skin expresses IL-1ra mainly in the differentiated stratum granulosum of the epidermis, whereas psoriatic skin expresses IL-1ra in basal and midbasal layers (Hammerberg et al, J. Clin. Invest. 90:571-583, 1992). Changes in the IL-1α:IL-1ra ratio in different strata of the epidermis may affect keratinocyte proliferation and differentiation. Chronic inflammatory bowel disease may also involve an altered IL-1:I1-1ra ratio since it is markedly increased in Crohn's disease and ulcerative colitis (Cominelli et al., Cytokine 6:A171, 1994). Experimental evidence also suggests that IL-1ra may be useful in chronic and acute cerebral neuropathologies (Relton et al., Exp. Neurol. 138:206-213, 1996; Loddick et al., Biochem. Biophys. Res. Comm. 234:211-215, 1997), insulin dependent diabetes mellitus (Madrup-Poulsen et al., Cytokine 5:185-191, 1993), glomerulonephritis (Lan et al., Kidney Int. 47:1303-1309, 1995), and pancreatitis (Norman et al., Ann. Surg. 221:625, 1995).

Recombinant IL-1ra has been shown to be well tolerated in clinical trials in humans (Campion et al., Arthritis and Rheumatism 39: 1092-1101, 1996), and to be potentially efficacious in the treatment of septic shock (Fisher et al., JAMA 271:1836-1843, 1994), rheumatoid arthritis (Campion et al., Arthritis & Rheumatism 39:1092-1101, 1996), and graft vs. host disease (GVHD) (Antin et al., Blood 84:1342-1348, 1994). However, high serum concentrations of the molecule are often required because of receptor binding affinity, plasma half-life, and tissue permeability.

The fibroblast growth factor (FGF) family consists of more than twenty distinct members (Basilico et al., Adv. Cancer Res. 59:115-165, 1992 and Femig et al., Prog. Growth Factor Res. 5(4):353-377, 1994), which bind heparin and generally act as mitogens for a broad spectrum of cell types. For example, basic FGF (also known as FGF-2) is mitogenic in vitro for endothelial cells, vascular smooth muscle cells, fibroblasts, and generally for cells of mesoderm or neuroectoderm origin, including cardiac and skeletal myocytes (Gospodarowicz et al., J. Cell. Biol. 70:395-405, 1976; Gospodarowicz et al., J. Cell. Biol. 89:568-578, 1981; and Kardami, J. Mol. Cell. Biochem. 92:124-134, 1990). In vivo, FGFs are believed to be important for the induction of angiogenesis, and bFGF has been shown to play a role in avian cardiac development (Sugi et al., Dev. Biol. 168:567-574, 1995 and Mima et al., Proc. Nat'l. Acad. Sci. 92:467-471, 1995) and to induce coronary collateral development in dogs (Lazarous et al., Circulation 94:1074-1082, 1996). In addition, non-mitogenic activities have been demonstrated for various members of the FGF family. Non-proliferative activities associated with acidic and/or basic FGF include increased endothelial release of tissue plasminogen activator, stimulation of extracellular matrix synthesis, chemotaxis of endothelial cells, induced expression of fetal contractile genes in cardiomyocytes (Parker et al., J. Clin. Invest. 85:507-514, 1990), and enhanced pituitary hormonal responsiveness (Baird et al., J. Cellular Physiol. 5:101-106, 1987). Several members of the FGF family do not have a signal sequence (aFGF, bFGF, and possibly FGF-9) and thus would not be expected to be secreted. In addition, several of the FGF family members have the ability to migrate to the cell nucleus (Friesel et al., FASEB 9:919-925, 1995).

There remains a need in the art for molecules that control inflammatory processes and for molecules that regulate cell division or metabolism.

### DESCRIPTION OF THE INVENTION

Within one aspect of the present invention there is provided an isolated protein comprising a sequence of amino acid residues as shown in SEQ ID NO:2 from residue 32 through residue 166. Within certain embodiments of the invention the protein comprises residues 32 through 170, residues 32 through 252, residues 3 through 166, residues 3 through 170, residues 3 through 252, residues 1 through 166, residues 1 through 170, or residues 1 through 252 of SEQ ID NO:2. Within other embodiments the protein is not more than 1500 amino acid residues in length. Within further embodiments the protein is not more than 500 amino acid residues in length. Within additional embodiments the protein further comprises an affinity tag or an immunoglobulin Fc region.

Within a second aspect of the invention there is provided an expression vector comprising the following operably linked elements: (a) a transcription promoter; (b) a DNA segment encoding a protein as disclosed above; and (c) a transcription terminator. Within one embodiment the expression vector further comprises a secretory signal sequence operably linked to the DNA segment.

Within a third aspect of the invention there is provided a cultured cell into which has been introduced the expression vector disclosed above, wherein the cell expresses the DNA segment.

Within a fourth aspect of the invention there is provided a method of making a protein comprising the steps of culturing a cell as disclosed above under conditions wherein the DNA segment is expressed and recovering the protein encoded by the DNA segment. Within one embodiment the expression vector further comprises a secretory signal sequence operably linked to the DNA segment, the protein encoded by the DNA segment is secreted into a culture medium in which the cell is cultured, and the protein is recovered from the culture medium.

Within a fifth aspect of the invention there is provided a protein produced according to the method disclosed above.

Within a sixth aspect of the invention there is provided an antibody that specifically binds to a protein as disclosed above.

Within a seventh aspect of the invention there is provided a method of modulating an immune response in an animal comprising administering to the animal a composition comprising a protein as disclosed above in combination with a pharmaceutically acceptable vehicle.

Within an eighth aspect of the invention there is provided a method of modulating the proliferation, differentiation, migration, or metabolism of mesencymal cells in an animal comprising administering to the animal a composition comprising a protein as disclosed above in combination with a pharmaceutically acceptable vehicle.

Within a ninth aspect of the invention there is provided a method of modulating the proliferation, differentiation, migration, or metabolism of mesencymal cells in culture comprising administering to cultured mesenchymal cells a composition comprising a protein as disclosed above in combination with a pharmaceutically acceptable vehicle.

These and other aspects of the invention will become evident upon reference to the following detailed description of the invention and the attached drawing.

The drawing is a Hopp/Woods hydrophilicity profile of the amino acid sequence shown in SEQ ID NO:2. The profile is based on a sliding six-residue window. Buried G, S, and T residues and exposed H, Y, and W residues were ignored. These residues are indicated in the figure by lower case letters.

The term “affinity tag” is used herein to denote a polypeptide segment that can be attached to a second polypeptide to provide for purification of the second polypeptide or provide sites for attachment of the second polypeptide to a substrate. In principal, any polypeptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985) (SEQ ID NO:9), substance P, Flag™ peptide (Hopp et al., Biotechnology 6:1204-1210, 1988), streptavidin binding peptide, maltose binding protein (Guan et al., Gene 67:21-30, 1987), cellulose binding protein, thioredoxin, ubiquitin, T7 polymerase, or other antigenic epitope or binding domain. See, in general, Ford et al., Protein Expression and Purification 2: 95-107, 1991. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequences. The term allelic variant is also used herein to denote a protein encoded by an allelic variant of a gene.

A “beta-strand” is a region of a protein characterized by certain combinations of the polypeptide backbone dihedral angles phi (φ) and psi (ψ). Regions wherein φ is less than −60° and ψ is greater than 90° are considered β-strands. Those skilled in the art will recognize that the limits of a β-strand are somewhat imprecise and may vary with the criteria used to define them. See, for example, Richardson and Richardson in Fasman, ed., Prediction of Protein Structure and the Principles of Protein Conformation, Plenum Press, New York, 1989; and Lesk, Protein Architecture: A Practical Approach, Oxford University Press, New York, 1991.

A “complement of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

“Conservative amino acid substitutions” are defined by the BLOSUM62 scoring matrix of Henikoff and Henikoff, Proc. Natl. Acad. Sci. USA 89:10915-10919, 1992, an amino acid substitution matrix derived from about 2,000 local multiple alignments of protein sequence segments, representing highly conserved regions of more than 500 groups of related proteins. As used herein, the term “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

The term “corresponding to”, when applied to positions of amino acid residues in sequences, means corresponding positions in a plurality of sequences when the sequences are optimally aligned.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include CDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In one form, an isolated polypeptide or protein is substantially free of other polypeptides or proteins, particularly other polypeptides or proteins of animal origin. In certain embodiments the polypeptides and proteins are provided in a highly purified form, i.e. greater than 95% pure or greater than 99% pure. When used in this context, the term “isolated” does not exclude the presence of the same polypeptide in alternative physical forms, such as dimers or alternatively glycosylated or derivatized forms.

A “motif” is a series of amino acid positions in a protein sequence for which certain amino acid residues are required. A motif defines the set of possible residues at each such position.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polynucleotide, polypeptide, or protein obtained from one species that is the functional counterpart of a polynucleotide, polypeptide, or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When these terms are applied to double-stranded molecules they are used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction. Many cell-surface receptors are, in their active forms, multi-subunit structures in which the ligand-binding and signal transduction functions may reside in separate subunits. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids, and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±20%.

All references cited herein are incorporated by reference in their entirety.

The present invention provides a group of novel proteins, designated “zil1a7”, that are structural homologs of the IL-1 and FGF families of cytokines. Analysis of the human zil1a7 sequence (SEQ ID NO:1 and NO:2) indicates that this protein, like other members of these families, contains a core structure of 12 β-strands wound into a β-barrel, with the β-strands separated from each other by loops. The loops between these β-strands are highly variable among the family members and are believed to be involved in receptor binding. These loops, which each contain at least three amino acid residues and may contain up to 17 residues, do not form β-strands or extended helices, but may (and often do) contain β-turns. Referring to SEQ ID NO:2, the twelve β-strands are formed by residues 23-27, 31-35, 51-54, 67-71, 79-83, 86-90, 95-98, 116-119, 128-132, 138-141, 148-150, and 159-163. These strands are characterized by a high content of hydrophobic amino acid residues (Leu, Val, Phe, and Ile). The loops include residues 28-30, 36-50, 55-66, 72-78, 84-85, 91-94, 99-115, 120-127, 133-137, 142-147, and 151-158. As noted above, this 12-stranded structure can be envisioned as three 4-stranded covalent monomers assembled into a “trefoil”. When these monomers are superimposed on each other, residues 66, 117, and 158 of SEQ ID NO:2 occupy like positions in their respective monomers.

In addition to the β-strands and loops, the zil1a7 proteins are characterized by the presence of conserved motifs at positions corresponding to (1) residues 130-134 of SEQ ID NO:2, (2) residues 94-98 of SEQ ID NO:2, (3) residues 156-160 of SEQ ID NO:2, and (4) residues 136-140 of SEQ ID NO:2. These motifs are shown in Table 1 using the standard single-letter codes for amino acid residues.

TABLE 1

| | | |
|---|---|---|
| | | SEQ ID |
| Motif | Sequence | NO: |

| | | |
|---|---|---|
| 1 | F/Y T/C/E/R S V/M/A/R A/Q/K | 3 |
| 2 | V/N/T/R L/I L/Q K/A/L E/S | 4 |
| 3 | I/G/V/A T/S/I D/G/K/L F Q/T/Y/L | 5 |
| 4 | P/G/T N/G/S L/P/W F/Y I/L/V | 6 |

The proteins of the present invention comprise residues 32 through 166 of SEQ ID NO:2. As disclosed in more detail below, these proteins can comprise additional amino acid residues derived from a zil1a7 protein or another protein. For example, additional residues N-terminal to residue 32 of SEQ ID NO:2 and/or residues C-terminal to residue 166 of SEQ ID NO:2 can be included. Representative proteins are shown in Table 2. Those skilled in the art will recognize that intermediate forms having amino and/or carboxyl termini between the illustrated positions (e.g., a carboxyl terminus at residue 170 or an amino terminus at residue 28 or residue 23) are also provided by the present invention.

```
TABLE 2
32-166
27-166
  3-166
  1-166
32-171
27-171
  3-171
  1-171
32-252
27-252
  3-252
  1-252
```

The proteins of the present invention are expected to have cytokine-like activity, such as immune-modulating or growth factor-like activity. Cytokine-like activities include, without limitation: regulation of cell division, including division of immune cells and mesenchymal cells (e.g., osteoblasts, chrondrocytes, adipocytes, muscle cells, fibroblasts, and endothelial cells), as well as precursors of these cells; regulation of vasculogenesis; regulation of angiogenesis; regulation of cell differentiation; stimulation of cell activation; and stimulation of cell migration. These activities are manifested within intact organisms as changes in cell populations, tissue structure, macromolecule production, biochemical markers, and other metabolic effects. While not wishing to be bound by theory, it is believed that zil1a7 proteins act through IL-1 or FGF receptors.

A limited number of non-conservative amino acid substitutions, amino acids that are not encoded by the genetic code, and non-naturally occurring amino acids may be incorporated into a zil1a7 protein. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-809, 1993; and Chung et al., Proc. Natl. Acad. Sci. USA 90:10145-10149, 1993). In a second method, translation is carried out in Xenopus oocytes by microinjection of mutated mRNA and chemically aminoacylated suppressor tRNAs (Turcatti et al., J. Biol. Chem. 271:19991-19998, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-7476, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

Those skilled in the art will recognize that additional amino acid sequence changes can be made in the zil1a7 sequence shown in SEQ ID NO:2 to obtain other zil1a7 proteins. These changes are made so as to minimize disruption of higher order structure essential to biological activity. In particular, the arrangement of β-strands and loops will not be disrupted, thus it is preferred to make conservative amino acid substitutions within the β-strands, particularly when replacing hydrophobic residues. Conservative amino acid substitutions are generally preferred. The effects of amino acid sequence changes can be predicted by computer modeling using available software (e.g., the Insight II® viewer and homology modeling tools; MSI, San Diego, Calif.) or determined by alignment and analysis of crystal structures. See, Priestle et al., EMBO J. 7:339-343, 1988; Priestle et al., Proc. Natl. Acad. Sci. USA 86:9667-9671, 1989; Finzel et al., J. Mol. Biol. 209:779-791, 1989; Graves et al., Biochem. 29:2679-2684, 1990; Clore and Gronenborn, J. Mol. Biol. 221:47-53, 1991; Vigers et al., J. Biol. Chem. 269:12874-12879, 1994; Schreuder et al., Eur. J. Biochem. 227:838-847, 1995; and Schreuder et al., Nature 386:194-200, 1997. A hydrophilicity profile of SEQ ID NO:2 is shown in the attached figure. Those skilled in the art will recognize that hydrophobicity and hydrophilicity will be taken into account when designing alterations in the amino acid sequence of a zil1a7 polypeptide, so as not to disrupt the overall profile.

The proteins of the present invention can further comprise amino- or carboxyl-terminal extensions, such as an amino-terminal methionine residue, an amino or carboxyl-terminal cysteine residue to facilitate subsequent linking to maleimide-activated keyhole limpet hemocyanin, a small linker peptide of up to about 20-25 residues, or an affinity tag as disclosed above. Two or more affinity tags may be used in combination. Polypeptides comprising affinity tags can further comprise a polypeptide linker and/or a proteolytic cleavage site between the zil1a7 polypeptide and the affinity tag. Exemplary cleavage sites include thrombin cleavage sites and factor Xa cleavage sites.

The present invention further provides a variety of other polypeptide fusions. For example, a zil1a7 polypeptide can be prepared as a fusion to a dimerizing protein as disclosed in U.S. Pat. Nos. 5,155,027 and 5,567,584. Dimerizing proteins in this regard include, for example, immunoglobulin fragments comprising constant region and hinge domains. For example, a zil1a7 polypeptide can be joined to an IgG Fc fragment (consisting essentially of C_{H}2, C_{H}3, and hinge). Such fusions are typically secreted as multimeric molecules wherein the Fc portions are disulfide bonded to each other and the two non-Ig polypeptides are arrayed in close proximity to each other. Dimerization can also be stabilized by fusing a zil1a7 polypeptide to a leucine zipper sequence (Riley et al., Protein Eng. 9:223-230, 1996; Mohamed et al., J. Steroid Biochem. Mol. Biol. 51:241-250, 1994). Immunoglobulin-zil1a7 polypeptide fusions and leucine zipper fusions can be expressed in genetically engineered cells to produce a variety of multimeric zil1a7 analogs. Auxiliary domains can be fused to zil1a7 polypeptides to target them to specific cells, tissues, or macromolecules (e.g., collagen). For example, a zil1a7 polypeptide or protein can be targeted to a predetermined cell type by fusing a zil1a7 polypeptide to a ligand that specifically binds to a receptor on the surface of the target cell. In this way, polypeptides and proteins can be targeted for therapeutic or diagnostic purposes. A zil1a7 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996. Within immunoglobulin-zil1a7 fusion proteins, certain amino acid subsititutions may be introduced into the Ig portion to alter effector functions associated with the native Ig. For example, amino acid substitutions can be made at EU index positions 234, 235, and 237 to reduce binding to FcγRI, and at EU index positions 330 and 331 to reduce complement fixation. See, Duncan et al., Nature 332:563-564, 1988; Winter et al., U.S. Pat. No. 5,624,821; Tao et al., J. Exp. Med. 178:661, 1993; and Canfield and Morrison, J. Exp. Med. 173:1483, 1991. The carboxyl-terminal lysine residue can be removed from the C_{H}3 domain to increase homogeneity of the product. Within fusions to an Ig heavy chain polypeptide, the Cys residue within the hinge region that is ordinarily disulfide-bonded to the light chain can be replaced with another amino acid residue, such as a serine residue, if the Ig fusion is not co-expressed with a light chain polypeptide. However, an Ig-zil1a7 fusion polypeptide can be co-expressed with a wild-type or fused light chain polypeptide as disclosed in U.S. Pat. No. 6,018,026. In addition, a zil1a7 polypeptide can be joined to another bioactive molecule, such as a cytokine, to provide a multi-functional molecule.

Polypeptide fusions of the present invention will generally contain not more than about 1,500 amino acid residues, often not more than about 1,300 residues, often not more than about 1,000 residues, and will in many cases be considerably smaller (e.g., 500 or fewer residues). For example, a zil1a7 polypeptide of 252 residues (residues 1-252 of SEQ ID NO:2) can be fused to E. coli β-galactosidase (1,021 residues; see Casadaban et al., J. Bacteriol. 143:971-980, 1980), a 10-residue spacer, and a 4-residue factor Xa cleavage site to yield a polypeptide of 1,287 residues. In a second example, residues 3-170 of SEQ ID NO:2 can be fused to maltose binding protein (approximately 370 residues), a 4-residue cleavage site, and a 6-residue polyhistidine tag. In a third example, residues 32 to 166 of SEQ ID NO:2 are fused at the C terminus to an IgG Fc fragment of 232 residues and at the N-terminus to a secretory peptide of 25 residues to yield a primary translation product of 392 residues and a processed polypeptide of 367 residues.

Essential amino acids in the proteins of the present invention can be identified according to procedures known in the art, such as site-directed mutagenesis or alanine-scanning mutagenesis (Cunningham and Wells, Science 244, 1081-1085, 1989; Bass et al., Proc. Natl. Acad. Sci. USA 88:4498-4502, 1991). In the latter technique, single alanine mutations are introduced at every residue in the molecule, and the resultant mutant molecules are tested for biological activity or other properties to identify amino acid residues that are critical to the activity of the molecule.

Multiple amino acid substitutions can be made and tested using known methods of mutagenesis and screening, such as those disclosed by Reidhaar-Olson and Sauer (Science 241:53-57, 1988) or Bowie and Sauer (Proc. Natl. Acad. Sci. USA 86:2152-2156, 1989). These authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WIPO Publication WO 92/06204), region-directed mutagenesis (Derbyshire et al., Gene 46:145, 1986; Ner et al., DNA 7:127, 1988), and DNA shuffling as disclosed by Stemmer (Nature 370:389-391, 1994) and Stemmer (Proc. Natl. Acad. Sci. USA 91:10747-10751, 1994). The resultant mutant molecules are tested for mitogenic activity or other properties (e.g., receptor binding) to identify amino acid residues that are critical to the activity of the molecule. Mutagenesis can be combined with high volume or high-throughput screening methods to detect biological activity of zil1a7 variant polypeptides, in particular biological activity in modulating cell proliferation or cell differentiation. For example, mitogenesis assays that measure dye incorporation or ³H-thymidine incorporation can be carried out on large numbers of samples. Mutagenized DNA molecules that encode active zil1a7 polypeptides can be recovered from host cells and rapidly sequenced using modern equipment. These methods allow the rapid determination of the importance of individual amino acid residues in a polypeptide of interest, and can be applied to polypeptides of unknown structure.

The present invention further provides polynucleotide molecules, including DNA and RNA molecules, encoding zil1a7 polypeptides. The polynucleotides of the present invention include the sense strand; the anti-sense strand; and the DNA as double-stranded, having both the sense and anti-sense strand annealed together by hydrogen bonds. A representative DNA sequence encoding a human zil1a7 protein is set forth in SEQ ID NO:1. DNA sequences encoding other zil1a7 proteins can be readily generated by those of ordinary skill in the art based on the genetic code. Counterpart RNA sequences can be generated by substitution of U for T.

Those skilled in the art will readily recognize that, in view of the degeneracy of the genetic code, considerable sequence variation is possible among these polynucleotide molecules. SEQ ID NO:7 is a degenerate DNA sequence that encompasses all DNAs that encode the zil1a7 polypeptide of SEQ ID NO:2. Those skilled in the art will recognize that the degenerate sequence of SEQ ID NO:7 also provides all RNA sequences encoding SEQ ID NO:2 by substituting U for T. Thus, zil1a7 polypeptide-encoding polynucleotides comprising nucleotide 94 to nucleotide 510 of SEQ ID NO:9 and their respective RNA equivalents are contemplated by the present invention, as are segments of SEQ ID NO:7 encoding other zil1a7 polypeptides disclosed herein. Table 3 sets forth the one-letter codes used within SEQ ID NO:7 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 3

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:7, encompassing all possible codons for a given amino acid, are set forth in Table 4.

TABLE 4

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | B | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by a degenerate sequence may encode variant amino acid sequences, but one of ordinary skill in the art can easily identify such variant sequences by reference to the amino acid sequence shown in SEQ ID NO:2. Variant sequences can be readily tested for functionality as described herein.

One of ordinary skill in the art will also appreciate that different species can exhibit preferential codon usage. See, in general, Grantham et al., Nuc. Acids Res. 8:1893-1912, 1980; Haas et al. Curr. Biol. 6:315-324, 1996; Wain-Hobson et al., Gene 13:355-364, 1981; Grosjean and Fiers, Gene 18:199-209, 1982; Holm, Nuc. Acids Res. 14:3075-3087, 1986; and Ikemura, J. Mol. Biol. 158:573-597, 1982. Preferred codons for a particular species can be introduced into the polynucleotides of the present invention by a variety of methods known in the art. Introduction of preferred codon sequences into recombinant DNA can, for example, enhance production of the protein by making protein translation more efficient within a particular cell type or species. Therefore, the degenerate codon sequence disclosed in SEQ ID NO:7 serves as a template for optimizing expression of polynucleotides in various cell types and species commonly used in the art and disclosed herein. Sequences containing preferred codons can be tested and optimized for expression in various host cell species, and tested for functionality as disclosed herein.

As previously noted, zil1a7 polynucleotides provided by the present invention include DNA and RNA. Methods for preparing DNA and RNA are well known in the art. In general, RNA is isolated from a tissue or cell that produces large amounts of zil1a7 RNA. Such tissues and cells are identified by conventional methods, such as Northern blotting (Thomas, Proc. Natl. Acad. Sci. USA 77:5201, 1980) or polymerase chain reaction (PCR; Mullis, U.S. Pat. No. 4,683,202). Total RNA can be prepared using guanidine-HCl extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zil1a7 polypeptides are then identified and isolated by, for example, hybridization or PCR.

The polynucleotides of the present invention can also be synthesized using automated equipment (“gene machines”) according to methods known in the art. See, for example, Glick and Pasternak, Molecular Biotechnology, Principles & Applications of Recombinant DNA, ASM Press, Washington, D.C., 1994; Itakura et al., Annu. Rev. Biochem. 53: 323-356, 1984; and Climie et al., Proc. Natl. Acad. Sci. USA 87:633-637, 1990.

The zil1a7 polynucleotide sequences disclosed herein can be used to isolate polynucleotides encoding other zil1a7 proteins. Such other polynucleotides include alternatively spliced cDNAs (including cDNAs encoding secreted zil1a7 proteins) and counterpart polynucleotides from other species (orthologs). These orthologous polynucleotides can be used, inter alia, as diagnostic reagents, experimental standards, and to prepare the respective orthologous proteins for use in veterinary medicine. For example, a cDNA can be cloned using mRNA obtained from a tissue or cell type that expresses zil1a7 as disclosed herein. Suitable sources of mRNA can be identified by probing Northern blots with probes designed from the sequences disclosed herein. A library is then prepared from mRNA of a positive tissue or cell line. A zil1a7-encoding cDNA can then be isolated by a variety of methods, such as by probing with a complete or partial human cDNA or with one or more sets of degenerate probes based on the disclosed sequences. Hybridization will generally be done under low stringency conditions, wherein washing is carried out in 1× SSC with an initial wash at 40° C. and with subsequent washes at 5° C. higher intervals until background is suitably reduced. A cDNA can also be cloned using by PCR using primers designed from the representative human zil1a7 sequence disclosed herein. Within an additional method, the cDNA library can be used to transform or transfect host cells, and expression of the cDNA of interest can be detected with an antibody to zil1a7 polypeptide.

Similar techniques can also be applied to the isolation of genomic clones. Genomic sequences are useful, for example, in producing zil1a7 proteins in transgenic animals. 5′ non-coding sequences of zil1a7 genes are also of interest, and zil1a7 polynucleotide sequences disclosed herein can be used as probes or primers to clone such 5′ non-coding regions. Promoter elements from a zil1a7 gene can be used to direct the expression of heterologous genes in, for example, transgenic animals or patients treated with gene therapy. Cloning of 5′ flanking sequences also facilitates production of zil1a7 proteins by “gene activation” as disclosed in U.S. Pat. No. 5,641,670. Briefly, expression of an endogenous zil1a7 gene in a cell is altered by introducing into the zil1a7 locus a DNA construct comprising at least a targeting sequence, a regulatory sequence, an exon, and an unpaired splice donor site. The targeting sequence is a zil1a7 5′ non-coding sequence that permits homologous recombination of the construct with the endogenous zil1a7 locus, whereby the sequences within the construct become operably linked with the endogenous zil1a7 coding sequence. In this way, an endogenous zil1a7 promoter can be replaced or supplemented with other regulatory sequences to provide enhanced, tissue-specific, or otherwise regulated expression. A portion of the human zil1a7 gene, including 5′ non-coding sequence, is shown in SEQ ID NO:8.

Those skilled in the art will recognize that the sequences disclosed in SEQ ID NO:1 and SEQ ID NO:2 represent a single allele of human zil1a7, and that natural variation, including allelic variation and alternative splicing, is expected to occur. Allelic variants of these sequences can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs that retain the inflammation modulating activity of zil1a7 are included within the scope of the present invention, as are polypeptides encoded by such cDNAs and mRNAs. The polypeptide sequence shown in SEQ ID NO:2 does not comprise a conventional cleavable secretory peptide. Many proteins, including members of the IL-1 family, are known to exist in both secreted and non-secreted forms. Alternatively spliced forms of zil1a7 comprising a secretory peptide may be expected to exist.

The polypeptides of the present invention, including full-length proteins and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, Green and Wiley and Sons, New York, 1993.

In general, a DNA sequence encoding a zil1a7 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zil1a7 polypeptide into the secretory pathway of a host cell, a secretory signal sequence (also known as a leader sequence, prepro sequence or pre sequence) is provided in the expression vector. The secretory signal sequence may be that of a zil1a7 gene, or may be derived from another secreted protein (e.g., t-PA) or synthesized de novo. The secretory signal sequence is operably linked to the zil1a7 DNA sequence, i.e., the two sequences are joined in the correct reading frame and positioned to direct the newly sythesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). In the alternative, a zil1a7 polypeptide is expressed cytoplasmically and is isolated after lysing the host cells.

Cultured mammalian cells are suitable hosts for use within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., ibid.), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters, such as promoters from SV-40 or cytomegalovirus, will be used. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” An exemplary selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that produce an altered phenotype, such as green fluorescent protein, or cell surface proteins such as CD4, CD8, Class I MHC, and placental alkaline phosphatase, can be used to sort transfected cells from untransfected cells by such means as FACS sorting or magnetic bead separation technology.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

Insect cells can be infected with recombinant baculovirus, commonly derived from Autographa californica nuclear polyhedrosis virus (AcNPV). See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and Richardson, Ed., Baculovirus Expression Protocols. Methods in Molecular Biology, Humana Press, Totowa, N.J., 1995. Recombinant baculovirus can also be produced through the use of a transposon-based system described by Luckow et al. (J. Virol. 67:4566-4579, 1993). This system, which utilizes transfer vectors, is commercially available in kit form (Bac-to-Bac™ kit; Life Technologies, Rockville, Md.). The transfer vector (e.g., pFastBac1™; Life Technologies) contains a Tn7 transposon to move the DNA encoding the protein of interest into a baculovirus genome maintained in E. coli as a large plasmid called a “bacmid.” See, Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a zil1a7-encoding sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses zil1a7 protein is subsequently produced. Recombinant viral stocks are made by methods commonly used the art.

For protein production, the recombinant virus is used to infect host cells, typically a cell line derived from the fall armyworm, Spodoptera frugiperda (e.g., Sf9 or Sf21 cells) or Trichoplusia ni (e.g., High Five™ cells; Invitrogen, Carlsbad, Calif.). See, in general, Glick and Pasternak, Molecular Biotechnology: Principles and Applications of Recombinant DNA, ASM Press, Washington, D.C., 1994. See also, U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (e.g., King and Possee, ibid.; O'Reilly et al., ibid.; Richardson, ibid.).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465, 1986; Cregg, U.S. Pat. No. 4,882,279; and Raymond et al., Yeast 14, 11-23, 1998. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808, 5,736,383, 5,854,039, and 5,888,768; and WIPO publications WO 99/14347 and WO 99/14320.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zil1a7 polypeptide in bacteria such as E. coli, the polypeptide may be retained in the cytoplasm or may be directed to the periplasmic space by a bacterial secretion sequence. In the former case, the cells are lysed, and the zil1a7 polypeptide is recovered from the lysate. If the polypeptide is present in the cytoplasm as insoluble granules, the cells are lysed, and the granules are recovered and denatured using, for example, guanidine isothiocyanate or urea. The denatured polypeptide can then be refolded by diluting the denaturant, such as by dialysis against a solution of urea and a combination of reduced and oxidized glutathione, followed by dialysis against a buffered saline solution. In the alternative, the protein may be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Zil1a7 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

The polypeptides of the present invention can be purified to ≧80% purity, ≧90% purity, ≧95% purity, or to a pharmaceutically pure state, that is greater than 99.9% pure with respect to contaminating macromolecules, particularly other proteins and nucleic acids, and free of infectious and pyrogenic agents.

Zil1a7 polypeptides (including fusion proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, N.Y., 1994. polypeptides comprising a polyhistidine affinity tag (typically about 6 histidine residues) are purified by affinity chromatography on a nickel chelate resin. See, for example, Houchuli et al., Bio/Technol. 6: 1321-1325, 1988. Polypeptides comprising a glu-glu tag can be purified by immunoaffinity chromatography according to conventional procedures. See, for example, Grussenmeyer et al., ibid. Maltose binding protein fusions are purified on an amylose column according to methods known in the art.

Using methods known in the art, zil1a7 polypeptides can be prepared in a variety of forms, including agonists and antagonists. The polypeptides may be pegylated or non-pegylated, and may or may not include an initial methionine amino acid residue. The actual structure of a recombinant zil1a7 polypeptide may depend in part on the host cell in which it is produced.

Zil1a7 proteins of the present invention may be used to modulate inflammation and related processes. Of particular interest is the reduction of inflammation and treatment of autoimmune diseases using forms of zil1a7 that act as IL-1 antagonists. Thus, certain zil1a7 proteins may be used to treat or prevent chronic inflammatory diseases such as arthritis (including rheumatoid arthritis, osteoarthritis, and Lyme arthritis) and psoriasis; to reduce tissue damage after ischemia; and to treat septic shock, graft-versus-host disease, and leukemia. As used herein, the terms “treat” and “treatment” will be understood to include the reduction of symptoms as well as effects on the underlying disease processes. Antagonists are expected to have in vivo activity like of that of IL-1 receptor antagonist (IL-1ra), which has shown beneficial effects in clinical trials directed to the treatment of rheumatoid arthritis (Campion et al., Arthritis & Rheumatism 39:1092-1101, 1996), graft-versus-host disease (Antin et al., Blood 84:1342-1348, 1994), septic shock (Fisher et al., JAMA 271:1836-1843, 1994), and leukemia (Dinarello, Blood 87:2095-2147, 1996). Experimental data also suggest that antagonism of IL-1 activity will prove beneficial in the treatment of inflammatory bowel disease (including Crohn's disease and ulcerative colitis) (reviewed by Hendel et al., Exp. Opin. Invest. Drugs 5:843-850, 1996; see also, Cominelli et al., Gastroenterology 103:65-71, 1992), insulin-dependent diabetes mellitus (reviewed by Mandrup-Poulsen et al., Cytokine 5:185-191, 1993; see also, Dayer-Metroz et al., Eur. J. Clin. Inv. 22:A50, 1992), acute pancreatitis (Norman et al., Ann. Surg. 221:625-634, 1995), glomerulonephritis (Lan et al., Kidney Int. 47:1303-1309, 1995), and cerebral ischemia (Relton et al., Exp. Neurology 138:206-213, 1996; Loddick et al., Biochem. Biophys. Res. Comm. 234:211-215, 1997).

Zil1a7 proteins may be used therapeutically to stimulate tissue development or repair, cell differentiation, or cell proliferation. Zil1a7 is expected to be useful for promoting the repair of soft and hard tissues, including skin, bone, ligament, and cartilage. Specific applications include, without limitation: the treatment of full-thickness skin wounds, including venous stasis ulcers and other chronic, non-healing wounds, particularly in cases of compromised wound healing due to diabetes mellitus, connective tissue disease, smoking, burns, and other exacerbating conditions; fracture repair, including non-union fractures and fractures in patients with compromised healing, such as diabetics, alcoholics, and the aged; bone grafts; healing bone following radiation-induced osteonecrosis; implants, including joint replacements and dental implants; repair of bony defects arising from surgery, such as cranio-maxiofacial repair following tumor removal, surgical reconstruction following tramatic injury, repair of hereditary or other physical abnormalities, and promotion of bone healing in plastic surgery; treatment of bone defects following therapeutic treatment of bone cancers; increase in bone formation during distraction osteogenesis; treatment of joint injuries, including repair of cartilage and ligament; repair of joints that have been afflicted with osteoarthritis; tendon repair and re-attachment; treatment of osteoporosis (including age-related osteoporosis, post-menopausal osteoporosis, glutocorticoid-induced osteoporosis, and disuse osteoporosis) and other conditions characterized by increased bone loss or decreased bone formation; elevation of peak bone mass in pre-menopausal women; use in the healing of connective tissues associated with dura mater; skin grafting; within reconstructive surgery to promote neovascularization and increase skin flap survival; to establish vascular networks in transplanted cells and tissues, such as transplanted islets of Langerhans; to treat female reproductive tract disorders, including acute or chronic placental insufficiency (an important factor causing perinatal morbidity and mortality) and prolonged bleeeding; to promote the growth of tissue damaged by periodontal disease and to repair other dental defects; to promote the repair of damaged liver tissue; in the treatment of acute and chronic lesions of the gastrointestinal tract, including duodenal ulcers, which are characterized by a deficiency of microvessels; to promote angiogenesis and prevent neuronal degeneration due to chronic cerebral ischemia; to accelerate the formation of collateral blood vessels in ischemic limbs; to promote vessel repair and development of collateral circulation following myocardial infarction so as to limit ischemic injury; to promote the repair of damaged cardiovascular tissue; to stimulate hematopoiesis, and to enhance T and B-cell function. The polypeptides are also useful additives in tissue adhesives for promoting revascularization of the healing tissue.

Inhibitors of zil1a7 activity (zil1a7 antagonists) may prove useful in the treatment of ocular neovascularization, including diabetic retinopathy and age-related macular degeneration. Experimental evidence suggests that these conditions result from the expression of angiogenic factors induced by hypoxia in the retina. Zil1a7 antagonists may also prove useful in the treatment of infantile hemangiomas, which exhibit overexpression of growth factors during the proliferative phase. Antagonists may also be used to limit the growth or metastasis of tumors. Anti-tumor activities may arise from reductions in a number of processes, including angiogenesis, lymphogenesis, and cell proliferation.

Biological activity of zil1a7 proteins is assayed using in vitro or in vivo assays designed to detect cytokine activity, in particular growth factor-like or immune-modulating activity. Many suitable assays are known in the art, and representative assays are disclosed herein. Assays using cultured cells are most convenient for screening, such as for determining the effects of amino acid substitutions, deletions, or insertions. However, in view of the complexity of developmental processes (e.g., angiogenesis and vasculogenesis), in vivo assays will generally be employed to confirm and further characterize biological activity. Certain in vitro models, such as gel matrix models, are sufficiently complex to assay histological effects. Assays can be performed using exogenously produced proteins (including zil1a7 fragments and fusion proteins), or may be carried out in vivo or in vitro using cells expressing the polypeptide(s) of interest. Representative assays are disclosed below.

Angiogenic effects of zil1a7 proteins can be measured using assays that are known in the art. Angiogenic activity of zil1a7 is indicated by an induction of angiogenesis or an associated biological response. For example, the effect of zil1a7 proteins on primordial endothelial cells in angiogenesis can be assayed in the chick chorioallantoic membrane angiogenesis assay (Leung, Science 246:1306-1309, 1989; Ferrara, Ann. NY Acad. Sci. 752:246-256, 1995). Briefly, a small window is cut into the shell of an eight-day-old fertilized egg, and a test substance is applied to the chorioallantoic membrane. After 72 hours, the membrane is examined for neovascularization. Other suitable assays include microinjection of early stage quail (Coturnix coturnix japonica) embryos as disclosed by Drake et al. (Proc. Natl. Acad. Sci. USA 92:7657-7661, 1995). Induction of vascular permeability, which is indicative of angiogenic activity, is measured in assays designed to detect leakage of protein from the vasculature of a test animal (e.g., mouse or guinea pig) after administration of a test compound (Miles and Miles, J. Physiol. 118:228-257, 1952; Feng et al., J. Exp. Med. 183:1981-1986, 1996). In vitro assays for angiogenic activity include the tridimensional collagen gel matrix model (Pepper et al. Biochem. Biophys. Res. Comm. 189:824-831, 1992 and Ferrara et al., Ann. NY Acad. Sci. 732:246-256, 1995), which measures the formation of tube-like structures by microvascular endothelial cells; and models employing a basement membrane matrix enriched in laminin (e.g., Matrigel®; Becton Dickinson, Franklin Lakes, N.J.) (Grant et al., “Angiogenesis as a component of epithelial-mesenchymal interactions” in Goldberg and Rosen, Epithelial-Mesenchymal Interaction in Cancer, Birkhäuser Verlag, 1995, 235-248; and Baatout, Anticancer Research 17:451-456, 1997), which are used to determine effects on cell migration and tube formation by endothelial cells seeded in the matrix.

Mitogenic activity can be measured using known assays, including ³H-thymidine incorporation assays (as disclosed by, e.g., Raines and Ross, Methods Enzymol. 109:749-773, 1985 and Wahl et al., Mol. Cell Biol. 8:5016-5025, 1988), dye incorporation assays (as disclosed by, for example, Mosman, J. Immunol. Meth. 65:55-63, 1983 and Raz et al., Acta Trop. 68:139-147, 1997), or cell counts. Suitable mitogenesis assays measure incorporation of ³H-thymidine into 20% confluent cultures or quiescent cells held at confluence for 48 hours. Suitable dye incorporation assays include measurement of the incorporation of the dye Alamar blue (Raz et al., ibid.) into target cells. See also, Gospodarowicz et al., J. Cell. Biol. 70:395-405, 1976; Ewton and Florini, Endocrinol. 106:577-583, 1980; and Gospodarowicz et al., Proc. Natl. Acad. Sci. USA 86:7311-7315, 1989.

Assays that can be used to measure zil1a7-induced production of one or more additional growth factors or other macromolecules are known in the art. Such assays include those for determining the presence of hepatocyte growth factor (HGF), epidermal growth factor (EGF), transforming growth factor alpha (TGFα), interleukin-6 (IL-6), VEGF, acidic fibroblast growth factor (aFGF), and angiogenin. Suitable assays include mitogenesis assays using target cells responsive to the macromolecule of interest, receptor-binding assays, competition binding assays, immunological assays (e.g., ELISA), and other formats known in the art. Metalloprotease secretion is measured from treated primary human dermal fibroblasts, synoviocytes and chondrocytes. The relative levels of collagenase, gelatinase and stromalysin produced in response to culturing in the presence of a zil1a7 protein is measured using zymogram gels (Loita and Stetler-Stevenson, Cancer Biology 1:96-106, 1990). Procollagen/collagen synthesis by dermal fibroblasts and chondrocytes in response to a test protein is measured using ³H-proline incorporation into nascent secreted collagen. ³H-labeled collagen is visualized by SDS-PAGE followed by autoradiography (Unemori and Amento, J. Biol. Chem. 265: 10681-10685, 1990). Glycosaminoglycan (GAG) secretion from dermal fibroblasts and chondrocytes is measured using a 1,9-dimethylmethylene blue dye binding assay (Farndale et al., Biochim. Biophys. Acta 883:173-177, 1986). Collagen and GAG assays are also carried out in the presence of IL-1β or TGF-β to examine the ability of zil1a7 protein to modify the established responses to these cytokines.

Effects of zil1a7 on bone cell growth and metabolism can be determined using known assays, including mitogenesis assays and assays of macromolecule producion, with bone-derived cells. Such cells include, for example, osteoblast cell lines, including Saos-2, a human primary osteogenic sarcoma cell line (ATCC No. HTB 85); U-2 OS, a human primary osteogenic sarcoma cell line (ATCC No. HTB 964); HOS (TE85), a human osteogenic sarcoma cell line (ATCC No. CRL 1543); MG-63, a human osteosarcoma cell line (ATCC No. CRL 1427); and UMR 106, a rat osteosarcoma cell line (ATCC No. CRL 1661).

Monocyte activation assays are carried out (1) to look for the ability of zil1a7 proteins to further stimulate monocyte activation, and (2) to examine the ability of zil1a7 proteins to modulate attachment-induced or endotoxin-induced monocyte activation (Fuhlbrigge et al., J. Immunol. 138: 3799-3802, 1987). IL-1β and TNFα levels produced in response to activation are measured by ELISA (Biosource, Inc. Camarillo, Calif.). Monocyte/macrophage cells, by virtue of CD14 (LPS receptor), are exquisitely sensitive to endotoxin, and proteins with moderate levels of endotoxin-like activity will activate these cells.

Cell differentiation can be assayed using suitable precursor cells that can be induced to differentiate into a more mature phenotype. For example, endothelial cells and hematopoietic cells are derived from a common ancestral cell, the hemangioblast (Choi et al., Development 125:725-732, 1998). Mesenchymal stem cells can also be used to measure the ability of zil1a7 protein to stimulate differentiation into osteoblasts. Differentiation is indicated by the expression of osteocalcin, the ability of the cells to mineralize, and the expression of alkaline phosphatase, all of which can be measured by routine methods known in the art. Effects of zil1a7 proteins or zil1a7 antagonists on tumor cell growth and metastasis can be analyzed using the Lewis lung carcinoma model, for example as described by Cao et al., J. Exp. Med. 182:2069-2077, 1995.

Assays for IL-1-like biological activity and receptor binding are known in the art. Exemplary activity assays include mitogenesis assays in which IL-1 responsive cells (e.g., D10.N4.M cells) are incubated in the presence of IL-1 or a test zil1a7 protein for 72 hours at 37° C. in a 5% CO₂ atmosphere. IL-2 (and optionally IL-4) is added to the culture medium to enhance sensitivity and specificity of the assay. [³H]thymidine is then added, and incubation is continued for six hours. The amount of label incorporated is indicative of agonist activity. See, Hopkins and Humphreys, J. Immunol. Methods 120:271-276, 1989; Greenfeder et al., J. Biol. Chem. 270:22460-22466, 1995. IL-1 stimulation of cell proliferation can also be measured using thymocytes cultured in IL-1 in combination with phytohemagglutinin. Proliferation is detected as [³H]thymidine incorporation or through the use of a colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). Briefly, a solution of MTT is added to 100 μl of assay cells, and the cells are incubated at 37° C. After 4 hours, 200 μl of 0.04 N HCl in isopropanol is added, the solution is mixed, and the absorbance of the sample is measured at 570 nm. Receptor binding can be measured by the competition binding method of Labriola-Tompkins et al., Proc. Natl. Acad. Sci. USA 88:11182-11186, 1991. Briefly, membranes pepared from EL-4 thymoma cells (Paganelli et al., J. Immunol. 138:2249-2253, 1987) are incubated in the presence of the test protein for 30 minutes at 37° C. Labeled IL-1α or IL-1β is then added, and the incubation is continued for 60 minutes. The assay is terminated by membrane filtration. The amount of bound label is determined by conventional means (e.g., γ counter). In an alternative assay, the ability of a zil1a7 protein to compete with labeled IL-1 for binding to cultured human dermal fibroblasts is measured according to the method of Dower et al. (Nature 324:266-268, 1986). Briefly, cells are incubated in a round-bottomed, 96-well plate in a suitable culture medium (e.g., RPMI 1640 containing 1% BSA, 0.1% Na azide, and 20 mM HEPES pH 7.4) at 8° C. on a rocker platform in the presence of labeled IL-1. Various concentrations of zil1a7 protein are added. After the incubation (typically about two hours), cells are separated from unbound label by centrifuging 60-μl aliquots through 200 μl of phthalate oils in 400-μl polyethylene centrifuge tubes and excising the tips of the tubes with a razor blade as disclosed by Segal and Hurwitz, J. Immunol. 118:1338-1347, 1977. Receptor binding can also be measured using immobilized receptors or ligand-binding receptor fragments. For example, an immobilized IL-1 receptor can be exposed to labeled IL-1 and unlabeled test protein, whereby a reduction in IL-1 binding compared to a control is indicative of receptor-binding activity in the test protein. Within another format, a receptor or ligand-binding receptor fragment is immobilized on a biosensor (e.g., BIACore™, Pharmacia Biosensor, Piscataway, N.J.) and binding is determined. Cloned cDNAs encoding mouse and human IL-1 receptors are disclosed by Dower et al., U.S. Pat. No. 5,081,228. IL-1 antagonists will exhibit receptor binding but will exhibit essentially no activity in IL-1 activity assays or will reduce the IL-1-mediated response when combined with IL-1. In view of the low level of receptor occupancy required to produce a response to IL-1, a large excess of antagonist (typically a 10- to 1000-fold molar excess) may be necessary to neutralize IL-1 activity.

The biological activities of zil1a7 proteins can be studied in non-human animals by administration of exogenous protein, by expression of zil1a7-encoding polynucleotides, and by suppression of endogenous zil1a7 expression through the use of inhibitory polynucleotides or knock-out techniques. Test animals are monitored for changes in such parameters as clinical signs, body weight, blood cell counts, clinical chemistry, histopathology, and the like. Animal models of angiogenesis, inflammatory disorders, autoimmune diseases, wound healing, tumor growth and metastasis, bone growth, and other cytokine-mediated processes are known in the art.

For assay of angiogenic activity, stimulation of coronary collateral growth can be measured in known animal models, including a rabbit model of peripheral limb ischemia and hind limb ischemia and a pig model of chronic myocardial ischemia (Ferrara et al., Endocrine Reviews 18:4-25, 1997). These models can be modified by the use of adenovirus or naked DNA for gene delivery as disclosed in more detail below, resulting in local expression of the test protein(s). Angiogenic activity can also be tested in a rodent model of corneal neovascularization as disclosed by Muthukkaruppan and Auerbach, Science 205:1416-1418, 1979, wherein a test substance is inserted into a pocket in the cornea of an inbred mouse. For use in this assay, proteins are combined with a solid or semi-solid, biocompatible carrier, such as a polymer pellet. Angiogenesis is followed microscopically. Vascular growth into the corneal stroma can be detected in about 10 days. Angiogenic activity can also be tested in the hampster cheek pouch assay (Hockel et al., Arch. Surg. 128:423-429, 1993). A test substance is injected subcutaneously into the cheek pouch, and after five days the pouch is examined under low magnification to determine the extent of neovascularization. Tissue sections can also be examined histologically. Induction of vascular permeability is measured in assays designed to detect leakage of protein from the vasculature of a test animal (e.g., mouse or guinea pig) after administration of a test compound (Miles and Miles, J. Physiol. 118:228-257, 1952; Feng et al., J. Exp. Med. 183:1981-1986, 1996).

Animal models of psoriasis include the analysis of histological alterations in adult mouse tail epidermis (Hofbauer et al, Brit. J. Dermatol. 118:85-89, 1988; Bladon et al., Arch Dermatol. Res. 277:121-125, 1985). In this model, anti-psoriatic activity is indicated by the induction of a granular layer and orthokeratosis in areas of scale between the hinges of the tail epidermis. Typically, a topical ointment is applied daily for seven consecutive days, then the animal is sacrificed, and tail skin is examined histologically. An additional model is provided by grafting psoriatic human skin to congenitally athymic (nude) mice (Krueger et al., J. Invest. Dermatol. 64:307-312, 1975). Such grafts have been shown to retain the characteristic histology for up to eleven weeks. As in the mouse tail model, the test composition is applied to the skin at predetermined intervals for a period of one to several weeks, at which time the animals are sacrificed and the skin grafts examined histologically. A third model has been disclosed by Fretland et al. (Inflammation 14:727-739, 1990). Briefly, inflammation is induced in guinea pig epidermis by topically applying phorbol ester (phorbol-12-myristate-13-acetate; PMA), typically at ca. 2 mg/ml in acetone, or the calcium ionophore A23187, typically at 200 nmol in 0.1 ml DMSO, to one ear and vehicle to the contralateral ear. Test compounds are applied concurrently with the PMA. Histological analysis is performed at 96 hours after application of PMA. This model duplicates many symptoms of human psoriasis, including edema, inflammatory cell diapedesis and infiltration, high LTB₄ levels, and epidermal proliferation.

Cerebral ischemia can be studied in a rat model as disclosed by Relton et al. (ibid.) and Loddick et al. (ibid.).

Wound-healing models include the linear skin incision model of Mustoe et al. (Science 237:1333, 1987). In a typical procedure, a 6-cm incision is made in the dorsal pelt of an adult rat, then closed with wound clips. Test substances and controls (in solution, gel, or powder form) are applied before primary closure. It is preferred to limit administration to a single application, although additional applications can be made on succeeding days by careful injection at several sites under the incision. Wound breaking strength is evaluated between 3 and 21 days after wounding. In a second model, multiple, small, full-thickness excisions are made on the ear of a rabbit. The cartilage in the ear splints the wound, removing the variable of wound contraction from the evaluation of closure. Experimental treatments and controls are applied. The geometry and anatomy of the wound site allow for reliable quantification of cell ingrowth and epithelial migration, as well as quantitative analysis of the biochemistry of the wounds (e.g., collagen content). See, Mustoe et al., J. Clin. Invest. 87:694, 1991. The rabbit ear model can be modified to create an ischemic wound environment, which more closely resembles the clinical situation (Ahn et al., Ann. Plast. Surg. 24:17, 1990). Within a third model, healing of partial-thickness skin wounds in pigs or guinea pigs is evaluated (LeGrand et al., Growth Factors 8:307, 1993). Experimental treatments are applied daily on or under dressings. Seven days after wounding, granulation tissue thickness is determined. This model is suitable for dose-response studies, as it is more quantitative than other in vivo models of wound healing. A full thickness excision model can also be employed. Within this model, the epidermis and dermis are removed down to the panniculus carnosum in rodents or the subcutaneous fat in pigs. Experimental treatments are applied topically on or under a dressing, and can be applied daily if desired. The wound closes by a combination of contraction and cell ingrowth and proliferation. Measurable endpoints include time to wound closure, histologic score, and biochemical parameters of wound tissue. Impaired wound healing models are also known in the art (e.g., Cromack et al., Surgery 113:36, 1993; Pierce et al., Proc. Natl. Acad. Sci. USA 86:2229, 1989; Greenhalgh et al., Amer. J. Pathol. 136:1235, 1990). Delay or prolongation of the wound healing process can be induced pharmacologically by treatment with steroids, irradiation of the wound site, or by concomitant disease states (e.g., diabetes). Linear incisions or full-thickness excisions are most commonly used as the experimental wound. Endpoints are as disclosed above for each type of wound. Subcutaneous implants can be used to assess compounds acting in the early stages of wound healing (Broadley et al., Lab. Invest. 61:571, 1985; Sprugel et al., Amer. J. Pathol. 129: 601, 1987). Implants are prepared in a porous, relatively non-inflammatory container (e.g., polyethylene sponges or expanded polytetrafluoroethylene implants filled with bovine collagen) and placed subcutaneously in mice or rats. The interior of the implant is empty of cells, producing a “wound space” that is well-defined and separable from the preexisting tissue. This arrangement allows the assessment of cell influx and cell type as well as the measurement of vasculogenesis/angiogenesis and extracellular matrix production.

One in vivo approach for assaying proteins of the present invention utilizes viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acids. For review, see Becker et al., Meth. Cell Biol. 43:161-89, 1994; and Douglas and Curiel, Science & Medicine 4:44-53, 1997. The adenovirus system offers several advantages. Adenovirus can (i) accommodate relatively large DNA inserts; (ii) be grown to high-titer; (iii) infect a broad range of mammalian cell types; and (iv) be used with many different promoters including ubiquitous, tissue specific, and regulatable promoters. Because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene is deleted from the viral vector, and the virus will not replicate unless the E1 gene is provided by the host cell (e.g., the human 293 cell line). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a signal sequence is present, secrete) the heterologous protein. Secreted proteins will enter the circulation in the highly vascularized liver, and effects on the infected animal can be determined.

In another embodiment, a zil1a7 gene can be introduced in a retroviral vector as described, for example, by Anderson et al., U.S. Pat. No. 5,399,346; Mann et al., Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO publication WO 95/07358; and Kuo et al., Blood 82:845, 1993. Alternatively, the vector can be introduced by “lipofection” in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages, including molecular targeting of liposomes to specific cells. Directing transfection to particular cell types is particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Within another embodiment target cells are removed from the animal, and the DNA is introduced as a naked DNA plasmid. The transformed cells are then re-implanted into the body of the animal. Naked DNA vectors can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Transgenic animals, engineered to express a zil1a7 gene, and animals that exhibit a complete absence of zil1a7 gene function, referred to as “knockout” animals (Snouwaert et al., Science 257:1083, 1992), can be generated (Lowell et al., Nature 366:740-42, 1993). See also, Brinster et al., Proc. Natl. Acad. Sci. USA 85: 836-840, 1988; Palmiter et al., Proc. Natl. Acad. Sci. USA 88: 478-482, 1991; Whitelaw et al., Transgenic Res. 1: 3-13, 1991; and WIPO publications WO 89/01343 and WO 91/02318). Polynucleotides used in generating transgenic animals that express a zil1a7 gene will preferably contain one or more introns; genomic sequences are thus preferred.

Zil1a7 inhibitory polynucleotides can be used to inhibit zil1a7 gene transcription or translation in a patient or test animal. Polynucleotides that are complementary to a segment of a zil1a7-encoding polynucleotide (e.g., a polynucleotide as set forth in SEQ ID NO:1) are designed to bind to zil1a7-encoding mRNA and to inhibit translation of such mRNA. Such antisense polynucleotides can be targetted to specific tissues using a gene therapy approach with specific vectors and/or promoters, such as viral delivery systems. Ribozymes can also be used as zil1a7 antagonists. Ribozymes are RNA molecules that contains a catalytic center and a target RNA binding portion. The term includes RNA enzymes, self-splicing RNAs, self-cleaving RNAs, and nucleic acid molecules that perform these catalytic functions. A ribozyme selectively binds to a target RNA molecule through complementary base pairing, bringing the catalytic center into close proximity with the target sequence. The ribozyme then cleaves the target RNA and is released, after which it is able to bind and cleave additional molecules. A nucleic acid molecule that encodes a ribozyme is termed a “ribozyme gene.” Ribozymes can be designed to express endonuclease activity that is directed to a certain target sequence in a mRNA molecule (see, for example, Draper and Macejak, U.S. Pat. No. 5,496,698, McSwiggen, U.S. Pat. No. 5,525,468, Chowrira and McSwiggen, U.S. Pat. No. 5,631,359, and Robertson and Goldberg, U.S. Pat. No. 5,225,337). An expression vector can be constructed in which a regulatory element is operably linked to a nucleotide sequence that encodes a ribozyme. In another approach, expression vectors can be constructed in which a regulatory element directs the production of RNA transcripts capable of promoting RNase P-mediated cleavage of mRNA molecules that encode a zil1a7 polypeptide. According to this approach, an external guide sequence can be constructed for directing the endogenous ribozyme, RNase P, to a particular species of intracellular mRNA, which is subsequently cleaved by the cellular ribozyme (see, for example, Altman et al., U.S. Pat. No. 5,168,053; Yuan et al., Science 263:1269, 1994; Pace et al., WIPO Publication No. WO 96/18733; George et al., WIPO Publication No. WO 96/21731; and Werner et al., WIPO Publication No. WO 97/33991). An external guide sequence generally comprises a ten- to fifteen-nucleotide sequence complementary to zil1a7 MRNA, and a 3′-NCCA nucleotide sequence, wherein N is preferably a purine. The external guide sequence transcripts bind to the targeted mRNA species by the formation of base pairs between the mRNA and the complementary external guide sequences, thus promoting cleavage of mRNA by RNase P at the nucleotide located at the 5′-side of the base-paired region.

For pharmaceutical use, the proteins of the present invention are formulated for local, including topical; or parenteral, including intravenous, subcutaneous, or intraperitoneal delivery according to conventional methods. Intravenous administration will be by injection or infusion. In many instances it will be beneficial to administer the protein by infusion or multiple injections per day over a period of several days to several weeks, sometimes preceded by a bolus injection. Other methods of sustained delivery, including controlled-release formulations, solid implants, and the like will be evident to those skilled in the art. In general, pharmaceutical formulations will include a zil1a7 protein in combination with a pharmaceutically acceptable vehicle, such as saline, buffered saline, 5% dextrose in water, or the like. Other suitable delivery vehicles include biocompatible solid or semi-solid matrices, including powdered bone, ceramics, biodegradable and non-biodegradable synthetic polymers, and natural polymers; tissue adhesives (e.g., fibrin-based); aqueous polymeric gels; lipid-based salves, ointments, and creams; liposomes; and the like. Exemplary formulations and delivery vehicles are disclosed below. This disclosure is illustrative; those skilled in the art will readily recognize suitable alternatives, including derivatives of the specifically named materials and combinations of materials. Formulations may further include one or more additional growth factors, excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. An “effective amount” of a composition is that amount that produces a statistically significant effect, such as a statistically significant increase in the formation of granulation tissue, increase in the rate of wound closure, increase in the rate of fracture repair, reduction or reversal of bone loss in osteoporosis, increase in the rate of healing of a joint injury, increase in the reversal of cartilage defects, increase or acceleration of bone growth into prosthetic devices, improved repair of dental defects, reduction of ischemia, increased vascularization of tissues, and the like. Zil1a7 will ordinarily be used in a concentration of about 10 to 100 μg/ml of total volume, although concentrations in the range of 1 ng/ml to 1000 μg/ml may be used. For local application, such as for dermal wound healing or the regeneration of bone in a fracture or other bony defect, the protein will be applied in the range of 0.1-100 μg/cm² of wound area. The exact dose will be determined by the clinician according to accepted standards, taking into account the nature and severity of the condition to be treated, patient traits, etc. Determination of dose is within the level of ordinary skill in the art.

Within the present invention zil1a7 can be used in combination with other cytokines and other therapeutic agents that have a positive effect on the condition to be treated. Such cytokines include insulin-like growth factor 1 (IGF-1), PDGF, alpha and beta transforming growth factors (TGF-α and TGF-β), epidermal growth factor (EGF), bone morphogenetic proteins, leukemia inhibitory factor, fibroblast growth factors, and IL-1ra. Other therapeutic agents include antibiotics, vitamin D, bisphosphonates, calcitonin, estrogens, parathyroid hormone, osteogenin, NaF, osteoprotegerin, and statins.

Zil1a7 can be delivered as a component of a tissue adhesive. Fibrin-based tissue adhesives are known in the art, and can be prepared from plasma or recombinant sources. Tissue adhesives comprise fibrinogen and factor XIII to which thrombin is added immediately before use to activate cross-linking. See, for example, Schwarz et al., U.S. Pat. No. 4,414,976; Stroetmann et al., U.S. Pat. No. 4,427,650; and Rose et al., U.S. Pat. No. 4,928,603. The use of tissue adhesives may be particularly advantageous in the treatment of conditions where connective tissue must be repaired, such as torn ligaments or tendons.

Solid and semisolid matrices are preferred delivery vehicles for filling non-union fractures, cavities, and other bony defects. These matrices provide a space-filling substitute for the natural bone, and include bone substituting agents such as tricalcium phosphate, hydroxyapatite, combinations of tricalcium phosphate and hydroxyapatite, polymethylmethacrylate, aluminates and other ceramics, and demineralized freeze-dried cortical bone. Solid and semi-solid matrices can also be prepared from a variety of polymeric materials. Semi-solid matrices provide the advantage of maleability such that they can be shaped to provide a precise filling of a bony defect. Matrices may include other active or inert components. Of particular interest are those agents that promote tissue growth or infiltration. Agents that promote bone growth include bone morphogenic proteins (U.S. Pat. No. 4,761,471; PCT Publication WO 90/11366), osteogenin (Sampath et al., Proc. Natl. Acad. Sci. USA 84: 7109-7113, 1987), and NaF (Tencer et al., J. Biomed. Mat. Res. 23: 571-589, 1989).

Biodegradable, synthetic polymers include polyesters, polyorthoesters, polyanhydrides, polycarbonates, polyfumarates, polyhydroxybutyrate, vinyl polymers, and the like. Specific examples include, without limitation, polylactide (PLA), polyglycolide (PGA), polylactide/polyglycolide copolymers, polydioxanone, polyglycolide/trimethylene carbonate copolymers, polyacrylic acid, polymethacrylic acid, polyvinyl pyrrolidone, and polyvinyl alcohol. Such materials can be prepared in a variety shapes, including films, plates, pins, rods, screws, blocks, lattices, and the like for attachment to or insertion into bone. See, for example, Walter et al., U.S. Pat. No. 5,863,297; and WIPO publication WO 93/20859. These materials may further include a carrier such as albumin, a polyoxyethylenesorbitan detergent or glutamic acid. In principle, any substance that enhances polymer degradation, creates pores in the matrix or reduces adsorption of the growth factor(s) to the matrix can be used as a carrier. Polyoxyethylenesorbitan detergents that are useful as carriers include polyoxyethylenesorbitan monooleate, polyoxyethylenesorbitan monolaureate, polyoxyethylenesorbitan monopalmitate, polyoxy-ethylenesorbitan monostearate and polyoxyethylenesorbitan trioleate. Plasticizers can also be included. Such polymers can be formed into pins, plates, screws, and the like and applied to the bone at a site of injury. Application is generally by implantation into the bone or attachment to the surface using standard surgical procedures. Polymer matrices are prepared according to procedures known in the art. See, for example, Loomis et al., U.S. Pat. No. 4,902,515; Gilding and Reed, Polymer 20: 1459-1464, 1979; and Boswell et al., U.S. Pat. No. 3,773,919.

Zil1a7 can also be delivered in combination with a biodegradable sponge, for example a gelatin, collagen, cellulose, or chitin sponge. Such sponges are known in the art. See, for example, Correll, U.S. Pat. No. 2,465,357; Miyata et al., U.S. Pat. No. 4,271,070; and Munck et al., WO 90/13320. A solution of zil1a7 and, optionally, one or more additional therapeutic agents is injected into the sponge, and the sponge is air-dried at a temperature of 30-100° C. for a time sufficient to reduce the water content to below 50%, preferably below 10%.

Gels can also be used as delivery vehicles. The use of aqueous, polymeric gels for the delivery of growth factors is disclosed by, for example, Finkenaur et al., U.S. Pat. No. 5,427,778; Edwards et al., U.S. Pat. No. 5,770,228; and Finkenaur et al., U.S. Pat. No. 4,717,717; and Cini et al., U.S. Pat. No. 5,457,093. Gels comprise biocompatible, water soluble or water swellable polymers that form viscous solutions in water. Such polymers include, without limitation, polysaccharides, including methyl cellulose, hydroxypropyl cellulose, hydroxypropylmethyl cellulose, hydroxyethyl cellulose, dextrans, starch, chitosan, and alginic acid; glycosaminoglycans, including hyaluronic acid, chondroitin, chondroitin sulfates, heparin, and heparan sulfate; proteins, including collagen, gelatin, and fibronectin; and acrylamides, including polyacrylamide and polymethacrylamide. Gels are generally prepared with a viscosity of from 200 cps to 100,000 cps, more commonly about 1000 cps to 30,000 cps at room temperature, the latter range corresponding to about 0.25-10% hydroxyethyl cellulose in water. Higher viscosity gels are known in the art (e.g., Finkenaur et al., U.S. Pat. No. 5,427,778). Viscosity can be adjusted by varying the concentration and/or length of the component polymer(s). Gels are prepared by combining the polymer with a suitable buffer, such as a low ionic strength citrate, phosphate, or acetate buffer at neutral or slightly acidic pH. A preservative (antimicrobial agent) such as methyl paraben, propyl paraben, benzyl alcohol, or the like, will generally be included. Following thorough mixing, the solution is sterilized by suitable means (e.g., autoclaving). The mixture is cooled, and filter-sterilized zil1a7 is added.

Alternative means for local delivery of zil1a7 include osmotic minipumps (e.g., ALZET® minipumps; Alza Corporation, Mountain View, Calif.); electrically charged dextran beads as disclosed in Bao et al. (WO 92/03125); collagen-based delivery systems, such as disclosed in Ksander et al. (Ann. Surg. 211:288-294, 1990); and alginate-based systems as disclosed in Edelman et al. (Biomaterials, 12:619-626, 1991). Other methods known in the art for sustained local delivery in bone include porous coated metal protheses that can be impregnated with a therapeutic agent and solid plastic rods with therapeutic compositions incorporated within them.

Zil1a7 may be used to promote the production of cartilage. Zil1a7 can be injected directly into the site where cartilage is to be grown. For example, zil1a7 can be injected directly in joints which have been afflicted with osteoarthritis or other injured joints in which the cartilage has been worn down or damaged by trauma. In the alternative, zil1a7 can be delivered in a suitable solid or semi-solid matrix as disclosed above.

Zil1a7 antagonists, including neutralizing antibodies, are formulated according to the general methods and principles disclosed above. As noted above, inhibition of IL-1 activity requires a large molar excess of antagonist. Doses of zil1a7 antagonist proteins will in general be quite large, particularly when treating life-threatening conditions. IL-1ra appears safe in high doses. Thus, doses of zil1a7 antagonist proteins will range from as low as 10 mg per patient per day to as high as 100 mg or more per hour infused over a period of days. Doses of IL-1ra found to be efficacious in clinical studies include 70 mg per patient per day in rheumatoid arthritis and up to 3,400 mg per patient per day in graft-versus-host disease. The exact dose will be determined by the clinician according to accepted standards, taking into account the nature and severity of the condition to be treated, patient traits, etc. Determination of dose is within the level of ordinary skill in the art. The proteins may be administered for acute treatment, over one week or less, but will often be used in treatment of chronic conditions requiring administration over several weeks to several months or longer. In general, a therapeutically effective amount of a zil1a7 protein is an amount sufficient to produce a clinically significant improvement in one or more standard indicators appropriate to the treated condition. Therapeutic endpoints will be apparent to those skilled in the art.

Zil1a7 proteins are also useful as laboratory reagents. For example, zil1a7 proteins can be used as cell culture components to promote cell growth or to expand selected cell populations. Those skilled in the art will recognize that zil1a7 proteins will often be used in combination with other cytokines. Other laboratory uses of zil1a7 proteins include use as molecular weight standards; as reagents in assays for determining levels of the protein in biological samples, such as in the diagnosis of disorders characterized by over- or under-production of zil1a7 protein; and as standards in the analysis of cell phenotype.

The invention further provides polypeptides that comprise an epitope-bearing portion of a protein as shown in SEQ ID NO:2. An “epitope” is a region of a protein to which an antibody can bind. See, for example, Geysen et al., Proc. Natl. Acad. Sci. USA 81:3998-4002, 1984. Epitopes can be linear or conformational, the latter being composed of discontinuous regions of the protein that form an epitope upon folding of the protein. Linear epitopes are generally at least 6 amino acid residues in length. Relatively short synthetic peptides that mimic part of a protein sequence are routinely capable of eliciting an antiserum that reacts with the partially mimicked protein. See, Sutcliffe et al., Science 219:660-666, 1983. Antibodies that recognize short, linear epitopes are particularly useful in analytic and diagnostic applications that employ denatured protein, such as Western blotting (Tobin, Proc. Natl. Acad. Sci. USA 76:4350-4356, 1979).

Antigenic, epitope-bearing zil1a7 polypeptides are useful for raising antibodies, including monoclonal antibodies, that specifically bind to a zil1a7 protein. The present invention thus provides polypeptides comprising at least 15 contiguous amino acid resdiues of SEQ ID NO:2. Polypeptides comprising a larger portion of a zil1a7 protein, e.g., at least 30, at least 50, at least 75, at least 100, at least 150, or at least 200 residues of SEQ ID NO:2 or up to the entire sequence are included. It is advantageous to select an amino acid sequence of the epitope-bearing polypeptide that provides substantial solubility in aqueous solvents, that is the sequence includes relatively hydrophilic residues, and hydrophobic residues are substantially avoided. Sequences containing proline residues are preferred. Preferred such regions include residues 26-31, 28-33, 45-50, 121-126, and 122-127 of SEQ ID NO:2. Somewhat longer peptides can be used as immunogens, such as a peptide comprising residues 26-54, 119-138, or 195-213 SEQ ID NO:2.

As used herein, the term “antibodies” includes polyclonal antibodies, monoclonal antibodies, antigen-binding fragments thereof such as F(ab′)₂ and Fab fragments, single chain antibodies, and the like, including genetically engineered antibodies. Non-human antibodies can be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. One skilled in the art can generate humanized antibodies with specific and different constant domains (i.e., different Ig subclasses) to facilitate or inhibit various immune functions associated with particular antibody constant domains. Alternative techniques for generating or selecting antibodies useful herein include in vitro exposure of lymphocytes to a zil1a7 protein, and selection of antibody display libraries in phage or similar vectors (for instance, through use of immobilized or labeled zil1a7 polypeptide). Antibodies are defined to be specifically binding if they bind to a zil1a7 protein with an affinity at least 10-fold greater than the binding affinity to control (non-zilla7) polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The affinity of a monoclonal antibody can be readily determined by one of ordinary skill in the art (see, for example, Scatchard, Ann. NY Acad. Sci. 51:660-672, 1949).

Methods for preparing polyclonal and monoclonal antibodies are well known in the art (see for example, Hurrell, J. G. R., Ed., Monoclonal Hybridoma Antibodies: Techniques and Applications, CRC Press, Inc., Boca Raton, Fla., 1982). As would be evident to one of ordinary skill in the art, polyclonal antibodies can be generated from a variety of warm-blooded animals such as horses, cows, goats, sheep, dogs, chickens, rabbits, mice, and rats. The immunogenicity of a zil1a7 protein may be increased through the use of an adjuvant such as alum (aluminum hydroxide) or Freund's complete or incomplete adjuvant. Polypeptides useful for immunization also include fusion polypeptides, such as fusions of a zil1a7 protein or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

A variety of assays known to those skilled in the art can be used to detect antibodies that specifically bind to a zil1a7 protein. Exemplary assays are described in detail in Antibodies: A Laboratory Manual, Harlow and Lane (Eds.), Cold Spring Harbor Laboratory Press, 1988. Representative examples of such assays include concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

Antibodies to zil1a7 may be used for affinity purification of zil1a7 proteins; within diagnostic assays for determining circulating levels of zil1a7 proteins; for detecting or quantitating soluble zil1a7 protein as a marker of underlying pathology or disease; for immunolocalization within whole animals or tissue sections, including immunodiagnostic applications; for immunohistochemistry; for screening expression libraries; and for other uses that will be evident to those skilled in the art. For certain applications, including in vitro and in vivo diagnostic uses, it is advantageous to employ labeled antibodies. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates.

Zil1a7 polynucleotides can be used to determine the presence of mutations at or near the zil1a7 locus at 8q23 in the human genome. Detectable chromosomal aberrations at the zil1a7 gene locus include, but are not limited to, aneuploidy, gene copy number changes, insertions, deletions, restriction site changes, and rearrangements. These aberrations can occur within the coding sequence, within introns, or within flanking sequences, including upstream promoter and regulatory regions, and may be manifested as physical alterations within a coding sequence or changes in gene expression level. Analytical probes will generally be at least 20 nucleotides in length, although somewhat shorter probes (14-17 nucleotides) can be used. PCR primers are at least 5 nucleotides in length, often 15 or more nt, more often 20-30 nt. Short polynucleotides can be used when a small region of the gene is targeted for analysis. For gross analysis of genes, a polynucleotide probe may comprise an entire exon or more. Probes will generally comprise a polynucleotide linked to a signal-generating moiety such as a radionucleotide. In general, these diagnostic methods comprise the steps of (a) obtaining a genetic sample from a patient; (b) incubating the genetic sample with a polynucleotide probe or primer as disclosed above, under conditions wherein the polynucleotide will hybridize to complementary polynucleotide sequence, to produce a first reaction product; and (c) comparing the first reaction product to a control reaction product. A difference between the first reaction product and the control reaction product is indicative of a genetic abnormality in the patient. Genetic samples for use within the present invention include genomic DNA, cDNA, and RNA. The polynucleotide probe or primer can be RNA or DNA, and will comprise a portion of SEQ ID NO:1, the complement of SEQ ID NO:1, or an RNA equivalent thereof. Suitable assay methods in this regard include molecular genetic techniques known to those in the art, such as restriction fragment length polymorphism 5 (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; A. J. Marian, Chest 108:255-65, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-38, 1991). The 8q23 region has been linked to a form of adult-onset open angle glaucoma (Trifan et al., Am. J. Opthal. 126:17-28, 1998).

Polynucleotides and polypeptides of the present invention will additionally find use as educational tools within laboratory practicum kits for courses related to genetics, molecular biology, protein chemistry, and antibody production and analysis. Due to their unique polynucleotide and polypeptide sequences, zil1a7 molecules can be used as standards or as “unknowns” for testing purposes. For example, zil1a7 polynucleotides can be used as aids in teaching a student how to prepare expression constructs for bacterial, viral, and/or mammalian expression, including fusion constructs, wherein a zil1a7 gene or cDNA is to be expressed; for determining the restriction endonuclease cleavage sites of the polynucleotides; determining mRNA and DNA localization of zil1a7 polynucleotides in tissues (e.g., by Northern blotting, Southern blotting, or polymerase chain reaction); and for identifying related polynucleotides and polypeptides by nucleic acid hybridization. Zil1a7 polypeptides can be used educationally as aids to teach preparation of antibodies; identification of proteins by Western blotting; protein purification; determination of the weight of expressed zil1a7 polypeptides as a ratio to total protein expressed; identification of peptide cleavage sites; coupling of amino and carboxyl terminal tags; amino acid sequence analysis, as well as, but not limited to, monitoring biological activities of both the native and tagged protein (e.g., receptor binding, signal transduction, proliferation, and differentiation) in vitro and in vivo. Zil1a7 polypeptides can also be used to teach analytical skills such as mass spectrometry, circular dichroism to determine conformation, in particular the locations of the disulfide bonds, x-ray crystallography to determine the three-dimensional structure in atomic detail, nuclear magnetic resonance spectroscopy to reveal the structure of proteins in solution, and the like. For example, a kit containing a zil1a7 polypeptide can be given to a student to analyze. Since the amino acid sequence would be known by the instructor, the polypeptide can be given to the student as a test to determine the skills or develop the skills of the student, and the instructor would then know whether or not the student has correctly analyzed the polypeptide. Since every polypeptide is unique, the educational utility of zil1a7 would be unique unto itself.

The invention is further illustrated by the following non-limiting examples.

### EXAMPLES

### Example 1

Recombinant human zil1a7 is produced in E. coli using a His₆ tag/maltose binding protein (MBP) double affinity fusion system as generally disclosed by Pryor and Leiting, Prot. Expr. Pur. 10:309-319, 1997. A thrombin cleavage site is placed at the junction between the affinity tag and zil1a7 sequences.

The fusion construct is assembled in the vector pTAP98, which comprises sequences for replication and selection in E. coli and yeast, the E. coli tac promoter, and a unique SmaI site just downstream of the MBP-His₆-thrombin site coding sequences. The zil1a7 cDNA (SEQ ID NO:1) is amplified by PCR using primers each comprising 40 bp of sequence homologous to vector sequence and 25 bp of sequence that anneals to the cDNA. The reaction is run using Pwo DNA polymerase (Boehringer Mannheim, Indianapolis, Ind.) for 30 cycles of 94° C., 30 seconds; 60° C., 60 seconds; and 72° C., 60 seconds. One microgram of the resulting fragment is mixed with 100 ng of SmaI-cut pTAP98, and the mixture was transformed into yeast to assemble the vector by homologous recombination (Oldenburg et al., Nucl. Acids. Res. 25:451-452, 1997). Ura⁺ transformants are selected.

Plasmid DNA is prepared from yeast transformants and transformed into E. coli MC1061. Pooled plasmid DNA is then prepared from the MC1061 transformants by the miniprep method after scraping an entire plate. Plasmid DNA is analyzed by restriction digestion using NcoI and EcoRI.

E. coli strain BL21 is used for expression of zil1a7. Cells are transformed by electroporation and grown on minimal glucose plates containing casamino acids and ampicillin.

Protein expression is analyzed by gel electrophoresis. Cells are grown in liquid medium containing ampicillin. After one hour at 37° C., IPTG is added to a final concentration of 1 mM, and the cells are grown for an additional 2-3 hours at 37° C. Cells are disrupted using glass beads, and extracts are prepared.

### Example 2

A mammalian cell expression vector encoding zil1a7 is constructed via homologous recombination. Zilla7 cDNA is isolated by PCR using primers that comprise, from 5′ to 3′ end, 40 bp of flanking sequence from the vector and 17 bp corresponding to the amino and carboxyl termini from the open reading frame of zil1a7. The resulting PCR product includes flanking regions at the 5′ and 3′ ends corresponding to the vector sequences flanking the zil1a7 insertion point. Ten μl of the 100 μl PCR reaction mixture is run on a 0.8% low-melting-temperature agarose (SeaPlaque GTG®; FMC BioProducts, Rockland, Me.) gel with 1× TBE buffer for analysis. The remaining 90 μl of the reaction mixture is precipitated with the addition of 5 μl 1 M NaCl and 250 μl of absolute ethanol.

The plasmid pZMP6, which has been cut with SmaI, is used for recombination with the PCR fragment. Plamid pZMP6 is a mammalian expression vector containing an expression cassette having the cytomegalovirus immediate early promoter, multiple restriction sites for insertion of coding sequences, a stop codon, and a human growth hormone terminator; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. It was constructed from pZP9 (deposited at the American Type Culture Collection, 10801 University Boulevard, Manassas, Va. 20110-2209, under Accession No. 98668) with the yeast genetic elements taken from pRS316 (deposited at the American Type Culture Collection, 10801 University Boulevard, Manassas, Va. 20110-2209, under Accession No. 77145), an internal ribosome entry site (IRES) element from poliovirus, and the extracellular domain of CD8 truncated at the C-terminal end of the transmembrane domain.

One hundred microliters of competent yeast (S. cerevisiae) cells are combined with 10 μl of the DNA preparations from above and transferred to a 0.2-cm electroporation cuvette. The yeast/DNA mixture is electropulsed using power supply (BioRad Laboratories, Hercules, Calif.) settings of 0.75 kV (5 kV/cm), ∞ ohms, 25 μF. To each cuvette is added 600 μl of 1.2 M sorbitol, and the yeast is plated in two 300-μl aliquots onto two URA-D plates and incubated at 30° C. After about 48 hours, the Ura⁺ yeast transformants from a single plate are resuspended in 1 ml H₂O and spun briefly to pellet the yeast cells. The cell pellet is resuspended in 1 ml of lysis buffer (2% Triton X-100, 1% SDS, 100 mM NaCl, 10 mM Tris, pH 8.0, 1 mM EDTA). Five hundred microliters of the lysis mixture is added to an Eppendorf tube containing 300 μl acid-washed glass beads and 200 μl phenol-chloroform, vortexed for 1 minute intervals two or three times, and spun for 5 minutes in an Eppendorf centrifuge at maximum speed. Three hundred microliters of the aqueous phase is transferred to a fresh tube, and the DNA is precipitated with 600 μl ethanol (EtOH), followed by centrifugation for 10 minutes at 4° C. The DNA pellet is resuspended in 10 μl H₂O.

Transformation of electrocompetent E. coli host cells (Electromax DH10B™ cells; obtained from Life Technologies, Inc., Gaithersburg, Md.) is done with 0.5-2 ml yeast DNA prep and 40 μl of cells. The cells are electropulsed at 1.7 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is plated in 250μl aliquots on four LB AMP plates (LB broth (Lennox), 1.8% Bacto™ Agar (Difco), 100 mg/L Ampicillin).

Individual clones harboring the correct expression construct for zil1a7 are identified by restriction digest to verify the presence of the zil1a7 insert and to confirm that the various DNA sequences have been joined correctly to one another. The inserts of positive clones are subjected to sequence analysis. Larger scale plasmid DNA is isolated using a commercially available kit (QIAGEN Plasmid Maxi Kit, Qiagen, Valencia, Calif.) according to manufacturer's instructions. The correct construct is designated pZMP6/zil1a7.

### Example 3

Full-length zil1a7 protein is produced in BHK cells transfected with pZMP6/zil1a7 (Example 2). BHK 570 cells (ATCC CRL-10314) are plated in 10-cm tissue culture dishes and allowed to grow to approximately 50 to 70% confluence overnight at 37° C., 5% CO₂, in DMEM/FBS media (DMEM, Gibco/BRL High Glucose; Life Technologies), 5% fetal bovine serum (Hyclone, Logan, Utah), 1 mM L-glutamine (JRH Biosciences, Lenexa, Kans.), 1 mM sodium pyruvate (Life Technologies). The cells are then transfected with pZMP6/zil1a7 by liposome-mediated transfection (using Lipofectamine™; Life Technologies), in serum free (SF) media (DMEM supplemented with 10 mg/ml transferrin, 5 mg/ml insulin, 2 mg/ml fetuin, 1% L-glutamine and 1% sodium pyruvate). The plasmid is diluted into 15-ml tubes to a total final volume of 640 μl with SF media. 35 μl of the lipid mixture is mixed with 605 μl of SF medium, and the resulting mixture is allowed to incubate approximately 30 minutes at room temperature. Five milliliters of SF media is then added to the DNA:lipid mixture. The cells are rinsed once with 5 ml of SF media, aspirated, and the DNA:lipid mixture is added. The cells are incubated at 37° C. for five hours, then 6.4 ml of DMEM/10% FBS, 1% PSN media is added to each plate. The plates are incubated at 37° C. overnight, and the DNA:lipid mixture is replaced with fresh 5% FBS/DMEM media the next day. On day 5 post-transfection, the cells are split into T-162 flasks in selection medium (DMEM+5% FBS, 1% L-Gln, 1% NaPyr, 1 μM methotrexate). Approximately 10 days post-transfection, two 150-mm culture dishes of methotrexate-resistant colonies from each transfection are trypsinized, and the cells are pooled and plated into a T-162 flask and transferred to large-scale culture.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated protein comprising a sequence of amino acid residues as shown in SEQ ID NO:2 from residue 32 through residue 166.

2. The isolated protein of claim 1 comprising a sequence of amino acid residues selected from the group consisting of:
residues 32 through 170 of SEQ ID NO:2;
residues 32 through 252 of SEQ ID NO:2;
residues 3 through 166 of SEQ ID NO:2;
residues 3 through 170 of SEQ ID NO:2;
residues 3 through 252 of SEQ ID NO:2;
residues 1 through 166 of SEQ ID NO:2;
residues 1 through 170 of SEQ ID NO:2; and
residues 1 through 252 of SEQ ID NO:2.

3. The isolated protein of claim 1 which is not more than 1500 amino acid residues in length.

4. The isolated protein of claim 1 which is not more than 500 amino acid residues in length.

5. The isolated protein of claim 1 further comprising an affinity tag.

6. The isolated protein of claim 1 further comprising an immunoglobulin Fc region.

7. An expression vector comprising the following operably linked elements:
(a) a transcription promoter;
(b) a DNA segment encoding a protein comprising a sequence of amino acid residues as shown in SEQ ID NO:2 from residue 32 through residue 166; and
(c) a transcription terminator.

8. The expression vector of claim 7 wherein the protein comprises a sequence of amino acid residues selected from the group consisting of:
residues 32 through 170 of SEQ ID NO:2;
residues 32 through 252 of SEQ ID NO:2;
residues 3 through 166 of SEQ ID NO:2;
residues 3 through 170 of SEQ ID NO:2;
residues 3 through 252 of SEQ ID NO:2;
residues 1 through 166 of SEQ ID NO:2;
residues 1 through 170 of SEQ ID NO:2; and
residues 1 through 252 of SEQ ID NO:2.

9. The expression vector of claim 7 wherein the protein is not more than 1500 amino acid residues in length.

10. The expression vector of claim 7 wherein the protein is not more than 500 amino acid residues in length.

11. The expression vector of claim 7 wherein the protein further comprises an affinity tag.

12. The expression vector of claim 7 wherein the protein further comprises an immunoglobulin Fc region.

13. The expression vector of claim 7 further comprising a secretory signal sequence operably linked to the DNA segment.

14. A cultured cell into which has been introduced the expression vector of claim 7, wherein the cell expresses the DNA segment.

15. A method of making a protein comprising:
culturing a cell according to claim 14 under conditions wherein the DNA segment is expressed; and
recovering the protein encoded by the DNA segment.

16. The method of claim 15 wherein the expression vector further comprises a secretory signal sequence operably linked to the DNA segment, wherein the protein encoded by the DNA segment is secreted into a culture medium in which the cell is cultured, and wherein the protein is recovered from the culture medium.

17. A protein produced according to the method of claim 15.

18. An antibody that specifically binds to the protein of claim 17.

19. A method of modulating an immune response in an animal comprising administering to the animal a composition comprising the protein of claim 17 in combination with a pharmaceutically acceptable vehicle.

20. A method of modulating the proliferation, differentiation, migration, or metabolism of mesencymal cells in an animal comprising administering to the animal a composition comprising the protein of claim 17 in combination with a pharmaceutically acceptable vehicle.

21. A method of modulating the proliferation, differentiation, migration, or metabolism of mesencymal cells in culture comprising administering to cultured mesenchymal cells a composition comprising the protein of claim 17 in combination with a pharmaceutically acceptable vehicle.


SEQUENCE LISTING (9 sequences)

SEQ ID NO: 1 - DNA, 759 bp, Homo sapiens
  CDS (1)...(759)
atgaaggttgcacaggcagagaagataaggttctcatattactccaacctccatctggatctgagctgctgggtcagtggagaggagcaccagcccaaactgatcccccagggctcttggttggaatcttcccagaagcctgaggggagagctgaagcacacctgtttcaaggaaaacagttggccaacaagacagcattggctttgtgggaatgtgccaaagaaatttcaagatattgtattcttttgattcatattcccaagttgctctccagaggaaatatactagcagaaggagagaatagcagccatttacagatagtggaatttggattatactcccacatgtacattcgaggccccgaaacgggggaagagaaaaaaatattttgcagtatggcctgtggcaatccttttcttcttgcttgctctgagtggtggctgctcagtggagggcccccagctggctctggtttccagctgcttcagagcccactggcctggagaaaaatctcatccagtcagattctcttgtcaggtgcatttgtaactggtgagaaccactcacaaacggcagtgcagggaatcctagctggtcgtgctgaccattcaggccaagagaatgtgccagccaggagagtttacctatacagtgacatcttgggtctcaggacagccccagttgtgctgagtgaagtcctcttaatgatctgtcacctcatggaagctttctcccaaggtccttcaccaaaacagtga

SEQ ID NO: 2 - protein, 252 aa, Homo sapiens
MKVAQAEKIRFSYYSNLHLDLSCWVSGEEHQPKLIPQGSWLESSQKPEGRAEAHLFQGKQLANKTALALWECAKEISRYCILLIHIPKLLSRGNILAEGENSSHLQIVEFGLYSHMYIRGPETGEEKKIFCSMACGNPFLLACSEWWLLSGGPPAGSGFQLLQSPLAWRKISSSQILLSGAFVTGENHSQTAVQGILAGRADHSGQENVPARRVYLYSDILGLRTAPVVLSEVLLMICHLMEAFSQGPSPKQ

SEQ ID NO: 3 - protein, 5 aa, Artificial Sequence
  peptide motif
XXSXX

SEQ ID NO: 4 - protein, 5 aa, Artificial Sequence
  peptide motif
XXXXX

SEQ ID NO: 5 - protein, 5 aa, Artificial Sequence
  peptide motif
XXXFX

SEQ ID NO: 6 - protein, 5 aa, Artificial Sequence
  peptide motif
XXXXX

SEQ ID NO: 7 - DNA, 756 bp, Artificial Sequence
  degenerate sequence
atgaargtngcncargcngaraarathmgnttywsntaytaywsnaayytncayytngayytnwsntgytgggtnwsnggngargarcaycarccnaarytnathccncarggnwsntggytngarwsnwsncaraarccngarggnmgngcngargcncayytnttycarggnaarcarytngcnaayaaracngcnytngcnytntgggartgygcnaargarathwsnmgntaytgyathytnytnathcayathccnaarytnytnwsnmgnggnaayathytngcngarggngaraaywsnwsncayytncarathgtngarttyggnytntaywsncayatgtayathmgnggnccngaracnggngargaraaraarathttytgywsnatggcntgyggnaayccnttyytnytngcntgywsngartggtggytnytnwsnggnggnccnccngcnggnwsnggnttycarytnytncarwsnccnytngcntggmgnaarathwsnwsnwsncarathytnytnwsnggngcnttygtnacnggngaraaycaywsncaracngcngtncarggnathytngcnggnmgngcngaycaywsnggncargaraaygtnccngcnmgnmgngtntayytntaywsngayathytnggnytnmgnacngcnccngtngtnytnwsngargtnytnytnatgathtgycayytnatggargcnttywsncarggnccnwsnccnaarcar

SEQ ID NO: 8 - DNA, 5044 bp, Homo sapiens
tgttatatcatgtctggatttaaaattaacagggactttttttgttgtggtaaaatatgcgtgtaaaatttaccattttagccatctttatgtgtacgtttcagttcactaagtacattcgcattgttgtgcaaccattactattgtacatctccagagctttttcatcttcccaagctgaaactccaaccccattaaactcactccctattcacccctccccatcagcccctgccaaccactgttcttctgtttttaaacagggacattttgagggaatttaaaataaacctgtccttcctcttctcttgcataagctattcaactaataaaatatctgttaaaagtcctaaattaattggaagtttcttaggtttaaacacatgggcaaaatgtacacaaaaaaataaacatctgtttcatgacagatgttttagacctacaacactgggaatgtgttcttgatgttaaaggaaaggagatggtaggctgaagcatgtgttccaaatcacttacttaacccagtgcaatttttatataatcactcaactaaccagccattcaacctgggagactttctagtcctagtaatatacccagatagtcttatgttagtcttctattcatattcattttaaataacttaagtatagaatcaatatatgtttatggttggacatttggaaaacacagaagaatatgaaaaagaaaagaaacatcattcttaatgttttactgtccaaagtaacaactactaatattttaaaattaaaaagcattacgtttaattttactttatttcaacagaagaaaaagaactctgaaatgaaattgaggatttgaacttcacacagctgtttgaagtaagagttactgtttctaactaattttaatttattaaaaattaaagatcaaactttaagagattgaggattaacttaaaaaaacattgtttcataatgagtacaggcttatatttttagttaataaagcttagttaactgtctcaggaagacagaaaagactgcagataatcaaacaaaaatacccttggttgggagaaggggttttaaaacagaattcaaaacctaatcccacctttaggattgttctcttggtgaaaattatataaaaaagggcaatccgtaaggaatatagttcatgaagtcgtaaaccactgcatctagtgaccttcccctttgaccagagaggcttaactttgtttgcttctaataccagtgttttttacattccacaaattgtttaaacacatactacattttagtttgctatatttgggccataattgccctttatgtatatttatttttctaaagtttctaattatctttgtcttctctgacctggagaaactaaaatcttcagtacatatgtggcatgtcatagtttaattgttggtgcttaattattagtcatggtaacctctttcttattgaagatgctctctactaagccctctgtgctaccatttaatgtggactagatttaagctcttctactagaatttctttccatcacaactggattattttcattgtctccacatctacccctttcttagactcattttcaattttgctagagcatatttttaatttttttcaaaataatagaagaagggctgtatctttgtagatcttcattttgccctcataattgaaaaatagacgccaaattactttccatcacaattttatggactttgtttcactgtcttctagcaacctggaaagcagatgcagagtctgctgccattttgattttgattcctttataggtagctccatcccccatccctctcccatgaacttttaggattttcttgttgtacttggaggtaataaattttaccaggaagtgtccagatattttttaaattattattcctcttcaggttttttaaatccaaagattcaaggtttattcagcccagaaaaatattcttctatgatttatttgaagtgttcatctctattttctttgttttctcctctagtagctcttactatggaaacgatactaggtcccttagctctaggagatgttaggtctcttggatctctcctctatggtcttaacttttttcccatattttctttcttcccttttatgtgcattcagaggattttctcaattttgtcttctggatctcaaatttatttttgagtcatttcatttcaattattcagactttccactgagcatctttgtaaaatcattttttatatttatgaatgtagacttttctctcaaatatccctaaaagtattactttaaaaaaaaaagttaccttctttgtcctttgttccaactccatttcctttagtatcagatgttctgttgctcttctggatccttctttcatatgttctatgttttccttcaataaccagtaacttttgactggcccaatcatatttataaatgaagggctgggttgactggcccaatcatatttatgaatgaacgactgtgctgactagtagaggtgactggagggatttctttggcagttacgtaggaatggttttccaccacacttctcttcctgaatggaaaggctggcagttcggctcacaggaggcacatgtgacagtgggaagcagacaggcaagcctgtgactgtcctgactgccaggaccaggaaggctttatttttagggatgaactacttttatgtactgcctttctgtacaaatcttaaaggggagtagaaagagatggaaaacaacattcttcatagtctttttattaattgccttgactgttccttctccctatggctccagacctgaattcttcagggttctgttgagaagaacagctctcatctcttcattagctctctctaaaatctcttttacattgtggtttttgttggctcaatccaaacatatctgttggctattttctataaatttcttaaaatttctgtctgctgatggtaacttttcttgatttccatcccagttcttgatttattctttttcatacacattttccaccacttagttggaatttttgaatgaagaataggcagactgtcatgtctagtctgccatcttgaagagagattttattaacatcagagtggctgccccagtgtgctgatgtatgattggcacgaggccatggtacatgccttactttttataaggctatttctccttctttcatggcccagacagcacccccaaccctgattgaccatatgatcaatatgggtcatttatacaaggagaacaaagtgaaaataggtgggtcacatgagccaattccattcatcagatccacaatatctgcctcaaccaggtactgtatcattcttcaggactccactgagtcccattcctaacttccccagtactcctgtcaaatacaaacctaaattagaccactaaccatacagttctacaattttctgtctgtcttctccattaagctacaagctttctgaatgtaaacatggtgtctcatttcatatctgtgtcctcagtgcaccccacggggactgggattaagtacccaacacagactgaataactcagggactgaatgaatgaacaaagaaatgagcaagcttgatgttgacctgggaaacaggtgtgttgtctttctttgctgatggggctagtgacctcctttttagtcatgtgaagctttggaggagggtgtttcaggcagaaggagcagtatggatgaaggcccagtggcaaactgggacatgtcttttggggggagactggtggaaattgttcccaaagggctgttgattattaattcatccaaagaaaatctctaatctctgttgtatcctcttctgcagagtggtagtctgactacagggttcttatctttgccagttcagtgagtgcaggtctaaatagagctgttttgttttagcagaaaggttgcgtccgcagttcaataggtgtcagtaaatgctctttcatggttctatataatcatgatttaaaacctacatcagtcttctttgcaaatgtacattaaagcatctgcttatacttgtattaagtagcattacttgtactaagtcctcacttcacagggacattatgaaatcacagacagcttagaatggagctttcaagttgtccttgggcccctgttgcctgctggaatggataaagcatgaccccctcagccaacaagctctgctgcttgttgtgatgtttggtagaacttccttgctccaatgcctccgactccagtcaccatctgaaagtcctccccagaggttggagtgctttgcctcctatttctgcaaacttaatattaaaaaaaaaattactggttgtcaaaaggaagcaggctgttaattcctggttaattgaaattagtcttgtaatttttagatgtgttattctattcacaaagcaggtacctgataagtgtttgtcagtgagtgaaagaatgaatgacacattagaggaaagactgggtttgtctttcctgaaattggagttaaggggcacatttgctgggaattatcccaggtaagcacagggatgtgcttttgatcacaggccatttgttctttccattttagccattttcagtctgatcattcatctcttgtctttgggaataattaatttcaggttttagaacattactaaagacatctcagtggagttcttttgtttgtctataacaggattgtctggggttcatgctgttcccaaggatctcactcctcacttgatgaggcagagaccctgactccaagggcgtacccatcacatccccaacttctcagcatcaaactgggtttgtttaagaatgaagccactagcattagaagtgtgacagtataagacaggatattctcagttgtatgactcaggagccaccttggcaaccatcagtgaaatgctaggttatcctaataggctagtgaggtcttggaatgctaagtgtaaagtcagtggcataaagggacacgttctcagcagcccttgatgaagtcaagactagggacaccaggagagaaatggctgca

SEQ ID NO: 9 - protein, 6 aa, Artificial Sequence
  peptide tag
EYMPME